(19) United States
(12) Patent Application Publication
(10) Pub. No.: US 2018/0243212 A1
(43) Pub. Date: Aug. 30, 2018

(54) TRANSDERMAL FORMULATIONS FOR DELIVERY OF DOXYCYCLINE, AND THEIR USE IN THE TREATMENT OF DOXYCYCLINE-RESPONSIVE DISEASES AND CONDITIONS

(71) Applicant: DELIVRA, INC., Burlington (CA)

(72) Inventors: Joseph Gabriele, Stoney Creek (CA); Mikaela Teris, Montreal (CA)

(21) Appl. No.: 15/757,012

(22) PCT Filed: Sep. 2, 2016
(86) PCT No.: PCT/CA2016/051047

§ 371 Date: Mar. 2, 2018

Related U.S. Application Data
(60) Provisional application No. 62/301,910, filed on Mar. 1, 2016.
(60) Provisional application No. 62/213,714, filed on Sep. 3, 2015.

(51) Int. Cl. A61K 9/107; A61K 9/00; A61K 31/65; A61K 31/198; A61K 47/22; A61K 9/06; A61K 9/70; A61K 9/08; A61K 47/40; A61K 47/24; A61P 17/02

(52) U.S. Cl. CPC A61K 9/107; A61K 9/0014; A61K 31/65; A61K 31/198; A61K 47/22; A61P 17/02; A61K 9/7023; A61K 9/08; A61K 47/40; A61K 47/24; A61K 9/06

(57) ABSTRACT

The present application is directed to transdermal formulations for the delivery of doxycycline compounds to a subject for the treatment of doxycycline-responsive diseases. In particular, the transdermal formulation is an emulsion comprising an oil phase, an aqueous phase and an external phase.

### CROSS-REFERENCE TO RELATED APPLICATIONS

This application claims the benefit of U.S. Provisional Patent Application Ser. Nos. 62/213,714, filed Sep. 3, 2015, and 62/301,910, filed Mar. 1, 2016, the contents of which are both hereby incorporated by reference in their entirety.

### FIELD

The present application relates to transdermal formulations for effective delivery of doxycycline and various methods of use thereof.

### BACKGROUND

Wound healing is a very orderly and highly controlled process characterized by four distinct but overlapping phases: hemostasis, inflammation, proliferation and remodeling (Diegelmann and Evans, 2004). The healing occurs as a cellular response to injury and involves activation of keratinocytes, fibroblasts, endothelial cells, macrophages, and platelets. Wound healing is known to require a balance between the accumulation of collagenous and non-collagenous extracellular matrix (ECM) components and their remodelling by matrix metalloproteinases (MMPs) and the tissue inhibitors of the MMPs (TIMPs) (Lobmann et al., 2002). In general, wound healing depends on several factors, including the patient's age and physical condition, the location of the wound, the cause of the injury, the accompanying diseases such as diabetes or renal insufficiency, which all have a negative effect on wound healing processes.

Diabetic individuals exhibit a documented impairment in the healing of acute wounds. Moreover, this population is prone to develop chronic non-healing diabetic foot ulcers (DFUs), which are estimated to occur in 15% of all persons with diabetes. DFUs are a serious complication of diabetes, and precede 84% of all diabetes related lower leg amputations (Brem and Tomic-Canic, 2007). The impaired healing of both DFUs and acute cutaneous wounds in persons with diabetes involves multiple complex physiological mechanisms. It has been hypothesized that diabetic foot injuries often fail to heal because persistently high concentrations of pro-inflammatory cytokines in the wound induce high concentrations of proteases, which degrade multiple growth factors, receptors and matrix proteins that are essential for wound healing (Mast and Schultz, 1996; Nath and Gulati, 1998; Neely et al., 2000; Lobmann et al., 2002).

In diabetic wounds, there is a disturbance in the expression and activation of MMPs, a group of enzymes responsible for ECM degradation (Lobmann et al., 2006). The degradation and remodeling of the ECM by MMPs is a key element of tissue repair (McCarty and Percival, 2013). The MMP levels in chronic wound fluid are almost 60 times higher than those in acute wounds. This increased protease activity supports tissue destruction and inhibits normal repair processes (Woo et al., 2007; Sibbald and Woo, 2008). Indeed, human diabetic wounds exhibit an excess of pro-inflammatory cytokines such as TNF-α, which contribute to an environment of increased protease activity in diabetic wounds (Stadekmann, 1998).

Wounds in diabetic patients typically show abnormal healing, characterized by chronicity, persistent inflammation, copious exudate, hyper-granulation, increased bacterial load, and reduced ability to heal (Falanga et al., 2006). Many chronic wounds fail to heal with conventional therapy, resulting in disability and impaired quality of life. New technologies using recombinant growth factors, autologous growth factors, or bioengineered skin-tissue substitutes have been shown to be effective, but these treatments are costly (Joyce et al., 2010). The long duration of treatment as well as high costs to treat the diabetic foot ulcers make it desirable to employ effective programs that prevent wounds from developing and accelerate healing rates once wounds occur (Williams and Armstrong, 1998). For example, there is a need for an effective, safe, and simple-to-use (such as topical application) agent that promotes healing by selectively reducing levels of pro-inflammatory cytokines (such as, TNF-α, interleukin-1, IL-6) and proteases.

Doxycycline (1, DOX), a semisynthetic, chemically modified, and Food and Drug Administration (FDA) approved tetracycline analog, is widely used to treat infections caused by both Gram-negative and Gram-positive microorganisms (James and Scott, 2012). In addition to its antibiotic properties, doxycycline is frequently used to treat Lyme disease, chronic prostatitis, sinusitis, pelvic inflammatory disease, malaria, scleroderma and skin diseases such as acne and rosacea.

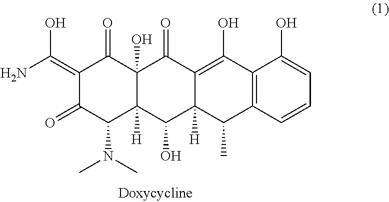 [SMILES: C[C@H]1c2cccc(O)c2C(O)=C2C(=O)[C@]3(O)C(=O)/C(=C(/N)O)C(=O)[C@@H](N(C)C)[C@@H]3[C@@H](O)[C@@H]21]

Biological Targets of Doxycycline

The tetracyclin analog was initially discovered in 1947 and has since been well-established that DOX is rapidly absorbed with a prolonged half-life and exerts biological effects independent of its antimicrobial activity (Golub et al., 1991). One such effect includes the inhibition of MMPs (Greenwald, 1994). MMPs are a family of zinc-dependent enzymes with the ability to degrade all components of the ECM. MMPs are produced by keratinocytes, endothelial cells, neutrophils, fibroblasts, macrophages, mast cells and eosinophils. There are three distinct subsets of enzymes that exist within the MMP family: collagenases, gelatinases, and stromelysins. Collagenases are the only enzyme in humans with the capacity to cleave the triple helix of type I, II, and III collagen (Mignatti et al., 1996).

Inhibition of MMP activity by DOX occurs through the chelation of calcium and zinc ions (Smith and Hasty, 2001). Doxycycline prevents the oxidative conversion of pro-MMPs in the ECM into active MMPs. This process is not dependent upon the metal-ion binding properties of DOX (Ryan and Baker, 2001).

Several animal studies reported, that treatments with DOX or other tetracycline analogous improved healing parameters (Chin et al., 2003). DOX also has shown potential to inhibit TNF-α converting enzyme (TACE) and prevent secretion of TNF-α into serum (Milano et al., 1997). Hence DOX can potentially promote dermal wound healing by reducing both protease activity and inflammation caused by TNF-α at the wound site (SivaNaga et al., 2011). Weckroth et al., (1996) reported that topical DOX reduced proteolytic activity in wound fluid of chronic venous leg ulcers. In addition, DOX has a long history as a collagenase inhibitor (James and Scott, 2012).

It has been reported that 1% DOX treatment for wound healing is beneficial due to reducing levels of TNF, MMPs, and elastase activities in the chronic wound environment, which improved the actions of endogenous growth factors (Chin et al., 2003). Therefore, DOX by means of its immune-modulatory and anti-inflammatory actions, through the inhibition of MMPs, could improve ECM functioning and represent a possible solution to support wound healing (Serra et al., 2015). Nonetheless, the instability problem with formulations of DOX is a major constraint to use the DOX for DFU treatment. Papich et al. (2013) reported that the concentration of DOX, compounded from commercial tablets in the vehicles cannot be assured beyond 7 days.

The premature metabolism of drugs as a result of the first-pass effect has made transdermal delivery an attractive and alternative strategy (Prausnitz, et al. 2008). For many years, people have placed natural substances on the skin for local ailments. However, lending this strategy towards all therapeutic drugs is not feasible. The human skin acts as a formidable barrier due in large part to the stratum comeum, which mostly consists of a lipid-enriched matrix and blocks entry of most topically applied agents, with the exception of low molecular weight, lipid-soluble drugs. This poses a challenge for administrating medications via the skin for either local cutaneous or systemic therapy.

Transdermal drug delivery strategies have thus focused primarily on the manipulation of this lipid milieu. In particular, penetration enhancers which interact with skin constituents to promote drug transport have provided an approach to increase the range of therapeutic agents that can be delivered.

Despite the significant permeability barrier of the stratum corneum, drug delivery via the skin is a very attractive option and is widely employed for both local and systemic therapy. Topical treatment of cutaneous disorders obviously targets the site of disease, thereby minimizing adverse side effects elsewhere within the body. Delivery of systemic therapies via the skin avoids degradation of the medication within the gastrointestinal tract and first-pass metabolism by the liver, both of which are associated with oral administration of drugs, in addition to evading the pain and safety issues associated with injections. Transdermal delivery of drugs, in some cases, enables infrequent dosing and maintenance of steady state drug levels.

Therefore, it is desirable to provide improved topical therapeutic compositions and delivery systems for the transdermal delivery of doxycycline across the dermis that could be used as a monotherapy or in conjunction with other agents to treat doxycycline-responsive diseases and/or conditions.

### SUMMARY

The present application includes transdermal formulations for the delivery of doxycycline to a subject. In some embodiments, the formulation comprises at least three phases including at least one oil phase, at least one aqueous phase and at least one external phase comprising doxycycline.

In some embodiments, the present application includes a transdermal formulation comprising:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient; wherein the oil and aqueous phase form an emulsion;
(c) an external phase comprising at least one flavonoid containing-extract, at least one phospholipid-complexed flavonoid, at least one antioxidant and a source of doxycycline; and optionally
(d) at least one preservative phase.

The present application includes methods for treating one or more doxycycline-responsive diseases and conditions comprising administering an effective amount of one or more of the transdermal formulations of the application to a subject in need thereof. In some embodiments, the doxycycline-responsive diseases and conditions are selected from one or more of Gram-negative and Gram-positive bacterial infections, skin diseases such as acne and rosacea, wrinkles, scleroderma, hyperpigmentation, Clostridium difficile colitis, early Lyme disease, malaria, chronic prostatitis, sinusitis and dermal wound healing.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows the stability of formulation 1 over 3 months at 45° C. for pH and viscosity evolution.

FIG. 2 shows the stability of formulation 2 over 3 months at 45° C. for pH and viscosity evolution.

FIG. 3 shows the stability of a second batch of formulation 2 over 3 months at 45° C. for pH and viscosity evolution.

FIG. 4 shows the stability of formulation 3 over 3 months at 45° C. for pH and viscosity evolution.

FIG. 5 illustrates a visual color comparison of doxycycline in formulations 1-3.

FIG. 6 is a graph illustrating the concentrations of doxycycline in formulations 1-3 on Jul. 30, 2015.

FIG. 7 is a graph illustrating the reduction of the wound surface area in patient 1 with complete healing by week 4.

FIG. 8 is a graph illustrating the reduction of the wound surface area in patient 2 with complete healing by week 4.

FIG. 9 is a graph illustrating the reduction of the wound surface area in patient 3 after 6 weeks.

FIG. 10 is a graph illustrating the reduction of the wound surface area in patient 4 with complete healing by week 4.

FIG. 11 is a graph illustrating the reduction of the wound surface area in patient 5 with complete healing by week 9.

FIG. 12 is a graph illustrating the reduction of the wound surface area in patient 6 after 6 weeks.

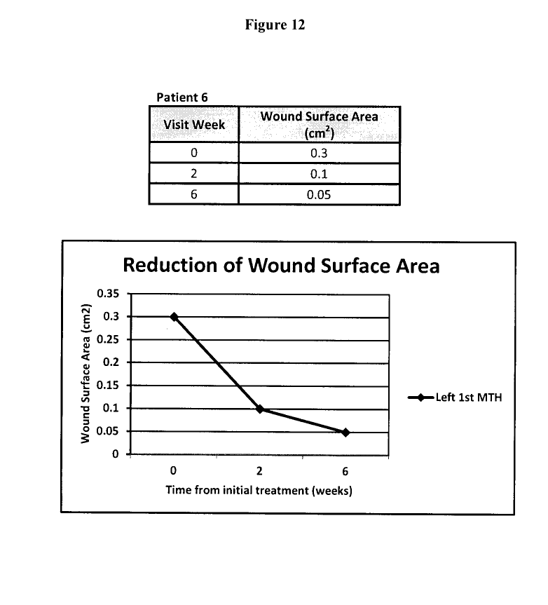

FIG. 13 is a graph illustrating the reduction of the wound surface area in patient 7 after 4 weeks.

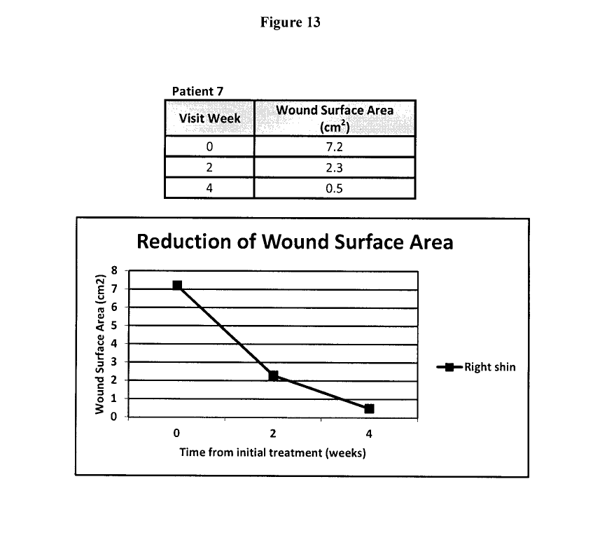

FIG. 14 is a graph illustrating the % initial doxycycline concentration remaining after 33 days at 5° C. and 25° C.

FIG. 15 is a graph illustrating the concentration of doxycycline in a formulation over a 27-day period.

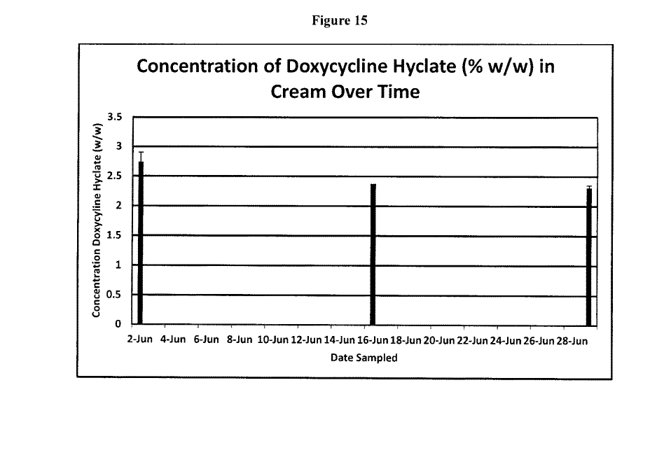

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The present description refers to a number of chemical terms and abbreviations used by those skilled in the art. Nevertheless, definitions of selected terms are provided for clarity and consistency.

As used in this application, the singular forms “a”, “an” and “the” include plural references unless the content clearly dictates otherwise. For example, an embodiment including “an agent” should be understood to present certain aspects with one compound or two or more additional compounds.

In embodiments comprising an “additional” or “second” component, such as an additional or second agent, the second component as used herein is chemically different from the other components or first component. A “third” component is different from the other, first, and second components, and further enumerated or “additional” components are similarly different.

The term “agent” as used herein indicates a compound or mixture of compounds that, when added to a formulation, tend to produce a particular effect on the formulation's properties.

The term “thickening agent” as used herein refers to a compound or mixture of compounds that adjusts the thickness of the formulation.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “water soluble”, for example as in “water soluble emulsion stabilizer”, refers to a substance that has a solubility in aqueous based solutions that is sufficient for the substance to exert its desired effect at concentrations that are pharmaceutically acceptable.

The term “oil soluble”, for example as in “oil soluble emulsion stabilizer”, refers to a substance that has a solubility in oil based solutions that is sufficient for the substance to exert its desired effect at concentrations that are pharmaceutically acceptable.

“Formulation” and “pharmaceutical formulation” as used herein are equivalent terms referring to a formulation for pharmaceutical use.

The term “pharmaceutically acceptable” means compatible with the treatment of animals, in particular, humans.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

“Topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

“Transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

“Transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The term “emollient” as used herein refers to a compound or mixture of compounds that adds or replaces natural oils in the skin, for example by maintaining the integrity of the hydrolipids of the skin.

The term “polar emollient” as used herein refers to emollient compounds, which are generally oils, having heteroatoms that differ in electronegativity. This results in a dipole moment. Typical polar oils are fatty alcohols, esters and triglycerides. While they are still water insoluble and oil-loving, these oils have unique characteristics due to their polar nature. They typically combine with higher hydrophobic lipid balance (HLB) emulsifiers to make stable emulsions, they dissolve materials that are insoluble in nonpolar oils, and they provide unique properties when compared with nonpolar oils such as mineral oil.

The term “medium polar emollient” as used herein refers to emollient compounds, which are generally oils that are less polar than the polar emollients but still more polar than nonpolar oils such as mineral oil.

The term “humectant” as used herein refers to a compound or mixture of compounds intended to increase the water content of the top layers of skin.

The term “emulsifier” of “emulsifying agent” as used herein refers to a compound of mixture of compounds which promote or facilitate the dispersion of one substance in another to form an emulsion.

The term “penetration enhancer” as used herein refers to a compound or mixture of compounds that improves the rate of percutaneous transport of an active agent across the skin for use and delivery of active agents to organisms such as mammals.

The term “preservative” as used herein refers to a substance that is added to products such as pharmaceutical compositions, to prevent decomposition by microbial growth or by undesirable chemical changes. For example, the addition of antimicrobial preservatives prevents microorganism growth by modifying the pH level.

The term “flavonoid compounds” as used herein refers to a class of plant secondary metabolites that have the general structure of a 15-carbon skeleton, which contains two phenyl rings (A and B) and heterocyclic ring (C). The basic chemical structure of a flavonoid as used herein is as follows:

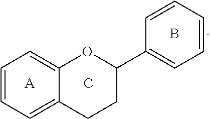

However, the term flavonoid includes the following flavonoids:

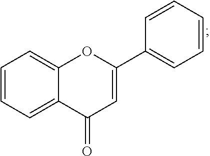 [SMILES: O=c1cc(-c2ccccc2)oc2ccccc12]

isoflavonoids:

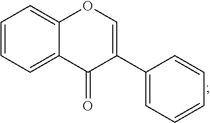 [SMILES: O=c1c(-c2ccccc2)coc2ccccc12]

and
neoflavonoids:

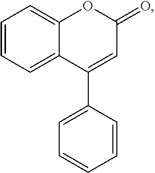 [SMILES: O=c1cc(-c2ccccc2)c2ccccc2o1]

as well as their non-ketone containing counterparts, known as flavanoids. Flavonoids are one of the largest known nutrient families, and include over 6,000 already-identified family members. Some of the best-known flavonoids include rutin, quercetin, kaempferol, catechins, and anthocyanidins. This nutrient group is most famous for its antioxidant and anti-inflammatory health benefits, as well as its contribution of vibrant color to foods.

The term “antioxidant” as used herein refers to molecules that inhibit the oxidation of other molecules, for example, by terminating chain reactions resulting from free radical intermediates and are often reducing agents.

The term “doxycycline” as used herein refers to compound belonging to a broad spectrum of antibiotics known as tetracycline antibiotics. Doxycycline has the following structure:

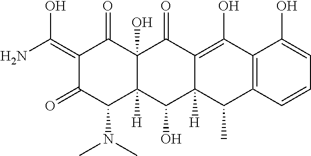 [SMILES: C[C@H]1c2cccc(O)c2C(O)=C2C(=O)[C@]3(O)C(=O)/C(=C(/N)O)C(=O)[C@@H](N(C)C)[C@@H]3[C@@H](O)[C@@H]21]

and includes tautomers thereof and pharmaceutically acceptable salts and solvates thereof.

The term “doxylcycline hyclate” as used herein refers to doxycycline hydrochloride hemiethanolate hemihydrate having the following structure:

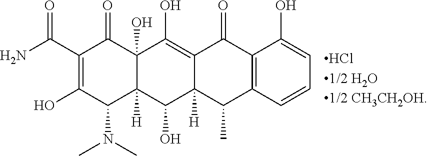 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

The term “pharmaceutically acceptable salt” means an acid addition salt or basic addition salt which is suitable for or compatible with the treatment of subjects, including human subjects.

The term “pharmaceutically acceptable acid addition salt” as used herein means a compound formed by the reaction of a pharmaceutically acceptable acid with a basic compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in either a hydrated, solvated or substantially anhydrous form. In general, the acid addition salts are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any pharmaceutically acceptable organic or inorganic base addition salt of any acid compound. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds for laboratory use, or for subsequent conversion to a pharmaceutically acceptable basic addition salt.

The term “wt %” means a percentage expressed in terms of weight of the ingredient or agent over the total weight of the formulation multiplied by 100.

The term “water” as used herein as an ingredient in the formulations of the application refers to pharmaceutically acceptable water.

### II. Formulations of the Application

In some embodiments, the transdermal formulation base of the present application comprises:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient; wherein the oil and aqueous phase form an emulsion;
(c) an external phase comprising at least one flavonoid containing-extract, at least one phospholipid-complexed flavonoid, at least one antioxidant and a source of doxycycline; and optionally
(d) at least one preservative phase.

In some embodiments, the transdermal formulation base comprises an oil-in-water emulsion. In some embodiments, the formulation is a multiphase emulsion, such as an oil-in-water-oil emulsion or a water-in-oil-water emulsion.

### Emulsifiers

In some embodiments the emulsifier is any oil-soluble fatty acid ester or mixture of fatty acid esters in which the fatty acid esters have a fatty acid composition similar to the fatty acid composition of skin for generating skin-compatible liquid crystals and to mimic the molecular organization of the intracellular lipidic laminae of the stratum corneum. Such liquid crystals are able to rapidly cross skin layers as well as to integrate into the skin's own lipid barrier to provide strength and greater integrity to this barrier.

In some embodiments the fatty acid esters are selected from sugar alcohol and fatty acid alcohol esters of any C₁₄-C₂₆-fatty acid or mixtures thereof. In some embodiments, the fatty acid esters are esters of fatty acids that are present in olive oil, palm oil and/or canola oil. In some embodiments, the fatty acids are esterified with fatty acid alcohols such as, but not limited to, cetyl alcohol, cetaryl alcohol, lauryl alcohol, stearyl alcohol, myristyl alcohol and/or oleyl alcohol. In some embodiments, the fatty acids are esterified with sugar alcohols such as, but not limited to, sorbitol, glycerol, mannitol, inositol, xylitol, erythritol, threitol, arabitol and/or ribitol. Olive oil fatty acid esters, and their use in transdermal formulations is described, for example, in U.S. Patent Application Publication No. 2011/0021439. In some embodiments, the fatty acid esters are sorbitan esters of palm oil or olive oil, such as sorbitan olivate or sorbitan palmitate. For example, sorbitan olivate is derived from fatty acids present in olive oil and esterified with sorbitol, and sorbitan palmitate is derived from fatty acids present in palm oil and esterified with sorbitol. In other embodiments, the fatty acid esters are cetearyl esters of olive oil, such as cetearyl olivate. For example, cetearyl olivate is derived from fatty acids present in olive oil and esterified with cetearyl alcohol. In further embodiments, the fatty acid esters are cetyl esters of palm oil, such as cetyl palmitate. For example, cetyl palmitate is derived from fatty acid esters present in palm oil and esterified with cetyl alcohol.

In some embodiments, the emulsifier is present in the formulations of the application in an amount of about 1 wt % to about 10 wt %, about 2 wt % to about 9 wt %, or about 3 wt % to about 5 wt %.

### Emulsion Stabilizers

In some embodiments, the emulsion stabilizer is any compound or mixture of compounds that helps to maintain the oil-in-water emulsion. There are three types of emulsion instability: flocculation, coalescence and creaming. Flocculation describes the process by which the dispersed phase comes out of suspension in flakes. Coalescence is another form of instability, which describes when small droplets combine to form progressively larger ones. Emulsions can also undergo creaming, which is the migration of one of the substances to the top or bottom (depending on the relative densities of the two phases) of the emulsion under the influence of buoyancy or centripetal force when a centrifuge is used. Generally, emulsion stability refers to the ability of an emulsion to resist change in its properties over time. In the present application an emulsion stabilizer is present in both the oil phase and the aqueous phase.

In some embodiments, the oil soluble emulsion stabilizer is one or more waxes. In some embodiments the waxes are selected from animal, plant and fruit waxes and mixtures thereof. In some embodiments, the plant wax is a wax derived from olives or from palm (e.g. carnauba wax). In some embodiments, the fruit wax is a wax derived from berries (e.g. berry wax). In some embodiment, the animal wax is beeswax. The one or more waxes are stabilizers that are present in the oil phase of the formulation.

In some embodiment, the oil soluble emulsion stabilizer is present in the formulation in an amount of about 0.5 wt % to about 5 wt % or about 1 wt % to about 4 wt %.

In some embodiments, the water soluble emulsion stabilizer is one or more thickening agents. In some embodiments, the thickening agents are any compound or mixture of compounds that maintains components in the formulation in suspension and provides a suitable consistency to the formulation.

In some embodiments, the water soluble emulsion stabilizer is selected from natural polymers, gums and synthetic polymers, and mixtures thereof. In some embodiments, natural polymers, gums and synthetic polymers, and mixtures thereof, are water soluble and therefore are present in the aqueous phase of the formulation. In some embodiments, the natural polymers are selected from alginic acid and derivatives thereof, cellulose and derivatives thereof and scleroglucans, and mixtures thereof. In some embodiments, the gums are selected from xanthan gum, tara gum, guar gum and arabic gum, and mixtures thereof. In some embodiments, the synthetic polymers are selected from polyacrylates, polyisobutenes and polysorbates, and mixtures thereof.

In some embodiments, the water soluble emulsion stabilizer is present in the formulations of the application in an amount of about 0.1 wt % to about 1 wt %, about 0.2 wt % to about 0.8 wt %, or about 0.3 wt % to about 0.7 wt %.

### Emollient Comprising at Least One Flavonoid

In some embodiments, the one or more emollients comprising one or more flavonoid compounds are polar emollients. Polar emollients generally include natural oils and extracts from plants. In some embodiments, the polar emollients are derived from fruits (including berries), vegetables, herbs, spices, legumes, leaves, seeds and/or grains. In some embodiments, the polar emollient is a natural oil or extract from citrus, Ginkgo biloba, tea, wine, cacao, onion, kale, parsley, red beans, broccoli, endive, celery, cranberries, blackberries, red raspberries, blackcurrants, acai, blueberries, bilberries, milk thistle, apples, hawthorn, Echinacea, grapes, and/or soy. In some embodiments, the polar emollient is emu oil

In some embodiments, the polar emollient comprising one or more flavonoid compounds is a natural oil or extract from the genera Rubus, Ribes, Argania, Nymphaea, Peucedanum or Imperatoria, Sambucus, Calendula, Butea, Citrus (e.g. lime), or species or subspecies thereof. In some embodiments, the polar emollient comprising one or more flavonoid compounds comprises Leptospermum Scoparium and/or manuka oil. In some embodiments, the polar emollient comprising one or more flavonoid compounds comprises Argan oil, Sea buckthorn oil, Cicatrol, Protectol, and/or Calendula.

In some embodiments, the emollients comprising one or more flavonoid compounds are present in the formulations of the application in an amount of about 1 wt % to about 20 wt %, about 2 wt % to about 10 wt %, or about 3 wt % to about 5 wt %.

### Further Emollients

The polarity of the emollients used in the present can vary depending on the identity of the emulsifiers and emulsion stabilizers, however can nonetheless be selected by a person skilled in the art. In some embodiments, the formulations of the present application comprise both polar emollients and medium polar emollients.

In some embodiments, further polar emollients used in the present application comprise an oil from an animal in the family Dromaius, for example Dromiceius (emu) or a plant, such as, Jojoba oil, Olive oil and/or coconut oil.

In some embodiments the one or more further polar emollients are present in an amount of about 0.5 wt % to about 10 wt %, about 1 wt % to about 7 wt %, or about 2 wt % to about 6 wt %.

In some embodiments, the medium polar emollient is an ester such as octyl palmitate, isopropyl stearate and isopropyl palmitate, or an alcohol such as octyl dodecanol, or mixtures thereof.

In some embodiments the emollients also act as a thickener (stabilizer) and/or a humectant.

In some embodiments, the one or more medium polar emollients are present in an amount of 0.5 wt % to about 10 wt %, about 1 wt % to about 7 wt %, or about 2 wt % to about 5 wt %.

### Flavonoid-Containing Extract

In some embodiments, the one or more flavonoid-containing extracts water phase is any suitable water soluble natural extract comprising a flavonoid with anti-inflammatory and/or antioxidant properties. In some embodiments, the one or more flavonoid-containing extracts are plant-based extracts, including but not limited to, one or more of Nymphaea caerulea flower extract, Peucedanum ostruthium leaf extract, Sambuscus nigra extract, Calendula flower Extract, Gingko biloba extract, Imperatoria Alpaflor extract, Sambucus Alpaflor extract, Blue lotus extract, Calendula Alpaflor extract, Masterwort extract, Elderberry extract, Angelica extract, green tea extract, chamomile extract, pomegranate pericarp and Peucedanum ostruthium leaf extract.

In some embodiments, the one or more flavonoid-containing extracts for the external phase are present in an amount of about 0.5 wt % to about 10 wt %, about 1 wt % to about 7 wt %, or about 2 wt % to about 5 wt %.

### Phospholipid-Complexed Flavonoid

In some embodiments, the flavonoid in the phospholipid-complexed flavonoid is a bioflavonoid isolated from plants such as, but not limited to, Gingko bilboa, Crataegus sp., Passiflora incarnata, Tormentilla potentilla, Tea sinensis., Aurantium sp., Citrus sp., Eucaliptus sp., Matricaria chamomilla, Rheum sp. and Fagara sylanthoides. In some embodiments, the flavonoid is isolated from green tea, buckwheat, the leaves and petioles of asparagus, fruit of the Fava D-Ante tree, fruits and fruit rinds, for example from citrus fruits such as orange, grapefruit, lemon and lime, and berries such as mulberries and cranberries. In some embodiments, the flavonoid is selected from quercetin, myrcetin, apigenin and rutin, and mixtures thereof.

In some embodiments, the phospholipid is any phospholipid, or mixture of phospholipids, from a plant or animal, or any synthetic phospholipid. In some embodiments, the phospholipid is selected from a phosphatidylcholine, a phosphatidylethanolamine, a phosphatidylinostinol, a phosphatidylserine and lecithin, and mixtures thereof.

In some embodiments, the phospholipid-complexed flavonoid is commercially available. In some embodiments, the phospholipid-complexed flavonoid is prepared by combining the phospholipid and flavonoid in a suitable solvent or mixture of solvents, in a mole ratio of phospholipid:flavonoid of about 0.5 to 2, or about 1, and isolating the resulting complex, for example, but removal of the solvent(s), precipitation and/or lyophilization.

In some embodiments, the phospholipid-complexed flavonoid is present in an amount of about 0.5% wt % to about 5 wt %, about 1 wt % to about 4 wt %, or about 1.5 wt % to about 2.5 wt %.

Complexes of bioflavonoids with phospholipids, their preparation and use, are described, for example in U.S. Pat. No. 5,043,323, the contents of which are incorporated by reference in their entirety.

### Source of Doxycycline

In some embodiments, the source of doxycycline is doxycycline hyclate. In some embodiments, the source of doxycycline is combined and/or dissolved in water prior to combining with the remaining ingredients of the formulation.

In some embodiments, the source of doxycycline is present in an amount of about 0.5 wt % to about 5 wt % or about 1 wt % to about 3 wt %. In some embodiments, the source of doxycycline is combined and/or dissolved in water which is used in an amount of about 2 wt % to about 10 wt % of the total formulation.

### Water

The balance of the aqueous phase of the composition is made up of water. Further, it is an embodiment that the solvent for the external phase, the source of doxycycline and/or the preservative phase (if present) comprises water. In some embodiments, the water is purified and/or demineralized water. The purified water may, for example, be filtered or sterilized.

In some embodiments, the amount of water in the aqueous phase is about 30 wt % to about 70 wt %, or about 40 wt % to about 65 wt % (based on the total weight of the formulation).

In some embodiments, the amount of water in the external phase is about 0.5 wt % to about 25 wt %, or about 1 wt % to about 20 wt % (based on the total weight of the formulation).

In some embodiments, the amount of water in the preservative phase (if present) is about 0 wt % to about 5 wt % (based on the total weight of the formulation).

In some embodiments, the amount of water used as the solvent for the source of doxycycline is about 2 wt % to about 10 wt % (based on the total weight of the formulation).

### Preservatives

In some embodiments, the formulations of the present application comprise at least one preservative. Preservatives include antimicrobial agents. In some embodiments the preservatives prevent or inhibit the growth of microorganisms, including bacteria, yeasts and molds. In some embodiments, the preservatives prevent or inhibit undesirable chemical reactions from occurring. For example, in some embodiments, the preservative is an antioxidant. In some embodiments, the preservative is an anti-bacterial or anti-fungal agent.

In some embodiments, the preservative comprises a preservative system comprising phenoxyethanol, benzoic acid, and dehydroacetic acid. In some embodiments, the preservative comprises capryl glycol, which also advantageously has humectant and emollient properties. In some embodiments, the preservative comprises chlorphenesin. In some embodiments, the preservative comprises ethylhexylglycerin which also advantageously has skin conditioning and emollient properties and acts as a deodorant. In some embodiments, the preservative comprises a natural antimicrobial agent (antibacterial, antifungal, antiviral). In some embodiments, the natural antimicrobial agent is selected from tea tree oil (Malaleuca alternifolia leaf oil) and myrtyl lemon essential oil. In some embodiments, the preservative comprises a preservative and a preservative booster.

In some embodiments, other components of the formulation have intrinsic anti-microbial properties.

In some embodiments, the one or more preservatives are present in an amount of about 0.01 wt % to about 5 wt %, about 1 wt % to about 4 wt %, or about 1.5 wt % to about 3 wt %.

### Antioxidants

It has been found that the formulations of the present application are susceptible to color degradation over time. Although the color degradation did not affect the efficacy of the formulations, the change in color from a light cream or beige color to a darker beige or brown color is undesirable. It was found that the addition of an antioxidant comprising an ascorbic acid (vitamin C) ester was particularly effective in reducing the color degradation of the formulations. Other known antioxidants, including for example a known Rosemary extract antioxidant, were not effective in reducing color degradation. In some embodiments, the antioxidant is selected from ascorbyl palmitate and ascorbyl stearate, and mixtures thereof.

In some embodiments, the one or more antioxidants are present in the formulation in an amount of about 0.01 wt % to about 2% wt or about 0.5 wt % to about 1.0 wt %.

In some embodiments, the one or more antioxidants are in the aqueous phase.

In some embodiments, the one or more antioxidants are in the external phase.

### Stabilizers

(a) Essential Minerals

Essential minerals are chemical elements needed for enzymatic reactions in the body, including the formation of healthy bones, protein and fatty acid formation and intestinal mobility. Non-limiting examples of essential minerals are calcium, phosphorous, potassium, sodium, chloride, magnesium and sulfur. Introducing essential materials into the human body is facilitated by carrier wherein the binding of the essential mineral to the carrier allows the essential mineral to be highly absorbable. In an embodiment of the application, the transdermal formulations further comprise essential minerals.

In some embodiments, the essential minerals are selected from one or more of magnesium citrate, magnesium taurate, magnesium glycinate, magnesium glutamate, magnesium aspartate, magnesium chloride, magnesium carbonate and magnesium sulfate. In some embodiments, the essential minerals are magnesium glycinate.

In some embodiments, the essential minerals are in the external phase.

(b) Cyclodextrin

Cyclodextrins are a family of cyclic oligosaccharides which are typically used in the food, pharmaceutical, chemical, environmental and agricultural industries to enhance the solubility and bioavailability of hydrophobic compounds as well as acting as stabilizers for volatile or unstable compounds.

In some embodiments, cyclodextrins include unfunctionalized and functionalized α-cyclodextrin, β-cyclodextrin and γ-cyclodextrin. In some embodiments, the transdermal formulations further comprise unfunctionalized and functionalized α-cyclodextrin, β-cyclodextrin and γ-cyclodextrin. In some embodiments, the cyclodextrin is selected from functionalized β-cyclodextrin. In a further embodiment, the cyclodextrin is 2-hydroxypropyl-β-cyclodextrin.

In some embodiments, the cyclodextrin is in the external phase.

### Further Optional Ingredients

In some embodiments, the formulations of the present application further comprise additional ingredients that are common in the transdermal base formulation art. These ingredients are, for example, but not limited to, further active pharmaceutical ingredients, pH adjusters or buffering agents, further solvents, solubilizers, chelating agents, pigments, fragrances, humectants, solubilizers, penetration enhancers, antioxidants and/or reducing agents.

(a) pH Adjusters/Buffering Agents

In some embodiments, the formulations of the application further comprise one or more pH adjusters, such as acidic, basic, or buffering components. These components may be added to provide the optimal pH balance for the skin. They may also be added to provide an optimal pH for one or more the components of the formulation. In some embodiments the pH of the formulations is adjusted to about 6 to about 7.5.

In some embodiments, the pH adjuster is selected from sodium hydroxide and potassium citrate. In some embodiment, the one or more pH adjusters are present in the formulation in an amount of about 0.05% wt % to about 2.0% wt, about 0.1 wt % to about 1.0 wt %, or about 0.8 wt % to about 0.8 wt %.

In some embodiments, the one or more pH adjusters are in the aqueous phase or the external phase.

(b) Chelating Agents

In some embodiments, the formulations of the application further comprise one or more chelating agents. In some embodiments, the chelating agents bind to metals which can inhibit the activity of the antimicrobial preservatives. In some embodiments, the chelating agent is sodium phytate or ethylendiamine tetraacetic acid (EDTA). In some embodiments, the one or more chelating agents are present in the formulation in an amount of about 0.01% wt % to about 0.2% wt, about 0.02 wt % to about 0.1 wt %, or about 0.03 wt % to about 0.05 wt %.

In some embodiments, the one or more chelating agents are in the aqueous phase or the external phase.

(c) Humectants

In some embodiments, the formulations of the present application further include one or more humectants. In some embodiments, the one or more humectants include, but are not limited to, glycerine (which also acts as an additional solvent).

In some embodiments, the one or more humectants are present in the formulation in an amount of about 0.5 wt % to about 10% wt, about 1 wt % to about 7 wt %, or about 2 wt % to about 5 wt %.

In some embodiments, the one or more humectants are in the aqueous phase.

(d) Solubilizers

In some embodiments, the formulations of the present application further include one or more solubilizers. In some embodiments, the one or more solubilizers include, but are not limited to, inulin lauryl carbamate.

In some embodiments, the one or more solubilizers are present in the formulation in an amount of about 0.01 wt % to about 5% wt.

In some embodiments, the one or more solubilizers are in the external phase.

(f) Further Active Ingredients

In some embodiments, the transdermal formulation of the present application further comprises other active ingredients. As used herein, active ingredients may include active molecules derived from natural, synthetic or semisynthetic means, as well as other active ingredients.

In some embodiments, the further active ingredient is solubilised or dispersed in an effective amount of a suitable vehicle (e.g. solvent(s) or diluent(s)). A skilled person can readily determine which solvents or diluents will be appropriate for a particular API.

In some embodiments, the further active ingredients are selected from compounds known to treat one or more doxycycline-responsive diseases and conditions. Examples of such compound are arginine and omithine and analogs thereof. In some embodiments, the L-arginine, L-ornithine or analogs thereof are included in the aqueous phase of the formulations of the application.

In some embodiments, the further API is included in an amount of about 0.01 wt % to about 1 wt %.

(g) Penetration Enhancer

In some embodiments the transdermal formulation of the present application further comprises penetration enhancers known in the art, for example, ethoxydiglycol (transcutanol), dimethyl isosorbide and mixtures thereof.

In some embodiments, the penetration enhancer is present in the formulation in an amount of about 0.5 wt % to about 10 wt %, or about 1 wt % to about 5 wt %.

In some embodiments, the transdermal formulation comprises:

(a) an aqueous phase comprising water, at least one thickening agent (such as xanthan gum), a humectant (such as glycerine) and optionally, a further active ingredient known to treat one or more doxycycline-responsive diseases and conditions (such as arginine);
(b) an oil phase comprising at least one emulsifier (such as cetearyl olivate, sorbitan olivate and wax stabilizers such as cetyl palmitate and sorbitan palmitate), at least one oil soluble emulsion stabilizer [wax emollient (such as carnauba wax)], at least one emollient comprising at least one flavonoid [such as natural oil or extract of red raspberries, blackcurrants and emu oil (polar emollient oils)], and at least one other emollient [polar emollient oil (such as isopropyl palmitate)]; wherein the oil and aqueous phase form an emulsion;
(c) an external phase comprising at least one flavonoid containing-extract (such as Imperatoria Alpaflor extract, Sambucus Alpaflor extract and Blue Lotus extract), at least one phospholipid-complexed flavonoid (such as lecithin and rutin), at least one anti-oxidant (such as ascorbyl palmitate), a source of doxycycline and optionally a penetration enhancer (such as transcutanol) and optionally a thickening agent (such as a mixture of synthetic polymers such as polyacrylates, polyisobutenes and polysorbate; and optionally
(d) at least one preservative phase (such as phenoxyethanol).

In some embodiments, the transdermal formulation comprises:

(a) an aqueous phase comprising water, at least one thickening agent (such as xanthan gum), a humectant (such as glycerine) and optionally, a further active ingredient known to treat one or more doxycycline-responsive diseases and conditions (such as arginine);
(b) an oil phase comprising at least one emulsifier (such as cetearyl olivate, sorbitan olivate and wax stabilizers such as cetyl palmitate and sorbitan palmitate), at least one oil soluble emulsion stabilizer [wax emollient (such as carnauba wax)], at least one emollient comprising at least one flavonoid [such as natural oil or extract of red raspberries, blackcurrants and emu oil (polar emollient oils)], and at least one other emollient [polar emollient oil (such as isopropyl palmitate)]; wherein the oil and aqueous phase form an emulsion;
(c) an external phase comprising at least one flavonoid containing-extract (such as Imperatoria Alpaflor extract, Sambucus Alpaflor extract and Blue Lotus extract), at least one phospholipid-complexed flavonoid (such as lecithin and rutin), at least one anti-oxidant (such as ascorbyl palmitate), a source of doxycycline, a cyclodextrine (such as β-cyclodextrine) and essential minerals (such as magnesium chloride or magnesium glycinate) and optionally a penetration enhancer (such as transcutanol) and optionally a thickening agent (such as a mixture of synthetic polymers such as polyacrylates, polyisobutenes and polysorbate; and optionally
(d) at least one preservative phase (such as phenoxyethanol).

In some embodiments, the formulations of the present application are prepared using a process that comprises:

(a) heating an aqueous phase comprising water and at least one water soluble emulsion stabilizer to a first temperature;
(b) heating an oil phase comprising at least one emulsifier, at least one oil soluble emulsion stabilizer, at least one emollient comprising at least one flavonoid, and at least one other emollient to the first temperature;
(c) adding the aqueous phase to the oil phase with stirring at the first temperature and continuing to stir at the first temperature until an emulsion is formed;
(d) cooling the emulsion in (c) to a second temperature; and, in any order:
(e) adding one or more external phases comprising at least one flavonoid containing-extract, at least one phospholipid-complexed flavonoid, at least one antioxidant and a source of doxycycline to the emulsion at the second temperature; and optionally
(f) adding one or more preservative phases to the emulsion.

In some embodiments, the first temperature is about 65° C. to about 85° C., about 70° C. to about 80° C., or about 75° C.

In some embodiments, the second temperature is about 30° C. to about 50° C., about 35° C. to about 45° C., or about 40° C.

In some embodiments, the process further comprises preparing the external phase wherein the at least one phospholipid-complexed flavonoid is stirred with water for a sufficient amount of time to become hydrated prior to being combined with the remaining ingredients for the external phase.

In some embodiments doxycycline is combined with a suitable solvent, such as water prior to being combined with the remaining ingredients for the external phase.

In some embodiments, the phases and emulsions are mixed with a homogenizer prior to combining with other phases.

In some embodiments, the phases and emulsions are mixed with a homogenizer prior to combining with other phases.

In some embodiments of the application the formulations described herein are in the form of a cream, gel, liquid suspension, ointment, solution, patch, film, foam or any other form for transdermal administration and the contents of the formulation adjusted accordingly. In some embodiments, the formulations are in the form of a cream. In some embodiments the cream has a viscosity of about 2000 cps to about 500000 cps, or about 5000 cps to about 200000 cps as measured using a Brookfield RVT T4-2, T4-10 or T4-100 RPM instrument at room temperature. In some embodiments, the formulations are applied as a foam, and subsequently, the area is bandaged to allow for transdermal administration.

In some embodiments of the application, the formulation maintains its initial color, pH and/or viscosity for at least one month, at least two months or at least three months.

In some embodiments, the formulations of the present application containing a source of doxycycline are stable for at least about 20 days, or at least about 30 days, at a temperature between about 0° C. to about 25° C. (room temperature), or about 0° C. to about 10° C., or about 4° C., wherein at least about 50%, or at least about 60%, or at least about 70%, or at least about 80%, of the doxycycline remains in the formulation after the time period. In some embodiments, after the storage period, not more than about 20% of the initial doxycycline decomposed to form decomposition products.

In other embodiments, the formulations of the present application containing a source of doxycycline, a cyclodextrine (such as β-cyclodextrine) and an essential mineral (such as magnesium chloride) are stable for at least 30 days, at temperature between about 0° C. to about 25° C. (room temperature), or about 0° C. to about 10° C., or about 5° C., wherein at least about 80%, at least about 90%, or at least about 95%, of the doxycycline remains in the formulation after the time period of at least 30 days.

### III. Methods of the Application

In some embodiments, the present application includes a method for transdermal administration of doxycycline comprising administering an effective amount of one or more of the formulations of the present application to a subject in need thereof. In further embodiments, the present application includes a use of one or more formulations of the present application for the administration of doxycycline to a subject.

The present application includes therapeutic methods and uses of the formulations described herein. In some embodiments, the formulations are used in methods to treat one or more doxycycline-responsive diseases and conditions.

Accordingly, the present application includes methods for treating one or more doxycycline-responsive diseases and conditions, comprising administering an effective amount of a transdermal formulation of the application to a subject in need thereof. Also included is a use of a transdermal formulation of the application to treat one or more doxycycline-responsive conditions. In some embodiments the doxycycline-responsive diseases and conditions are selected from one or more of Gram-negative and Gram-positive bacterial infections, skin diseases such as acne and rosacea, wrinkles, scleroderma, hyperpigmentation, Clostridium difficile colitis, early Lyme disease, malaria, chronic prostatitis, sinusitis and dermal wound healing. In some embodiments, the transdermal formulations of the application are to treat chronic venous leg ulcers or diabetic foot ulcers.

In some embodiments, the formulations of the application are used in conjunction with other therapies to treat doxycycline-responsive diseases and conditions.

### EXAMPLES

The following non-limiting examples are illustrative of the present application:

### Example 1: Preparation of Exemplary Transdermal Base Formulations Containing Doxycycline Source

Topical formulations comprising doxycycline were prepared using the ingredients listed in Tables 1, 3, 6 and 11.

### Procedure for Making Formulations 1-4

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase D were combined.

Step D: In a stainless steel container, ingredients of Phase E were combined.

Step E: While stirring, mixtures from steps C-D were added to the mixture from step B along with the preservative of Phase C. The combined solution mixtures were stirred until homogenous. The resulting solution mixture was stirred until homogenous and then cooled to room temperature.

### Storage Stability of Formulation 1.

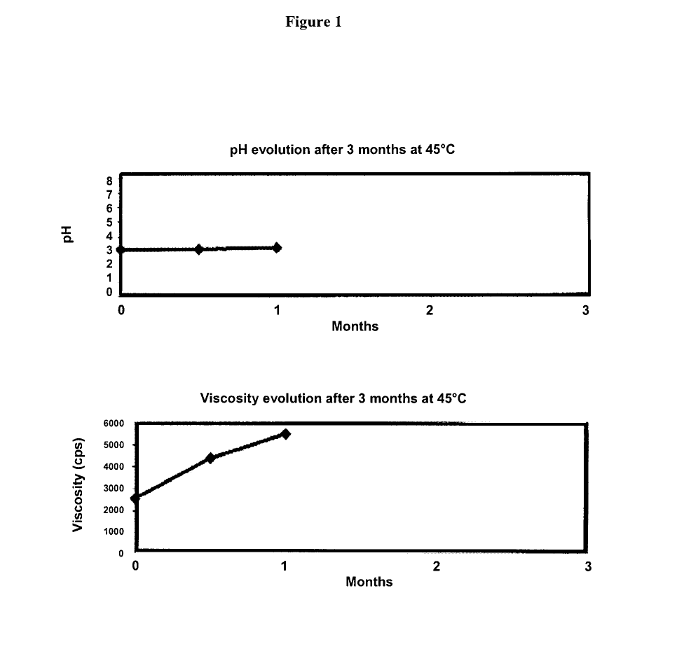

Formulation 1 was evaluated for its stability using four parameter measurements which included pH, texture, color and odour. Formulation 1 was stable for at least 1 month whereby the formulation provided an average pH evolution of 3.19±0.05 with a consistent viscosity evolution averaging at 4132 cps±1470 as depicted in both FIG. 1 and Table 2. The color of formulation 1 remained a light beige during the one month testing period and for an additional 2 months following the pH and viscosity testing (data not shown).

### Storage Stability of Formulation 2.

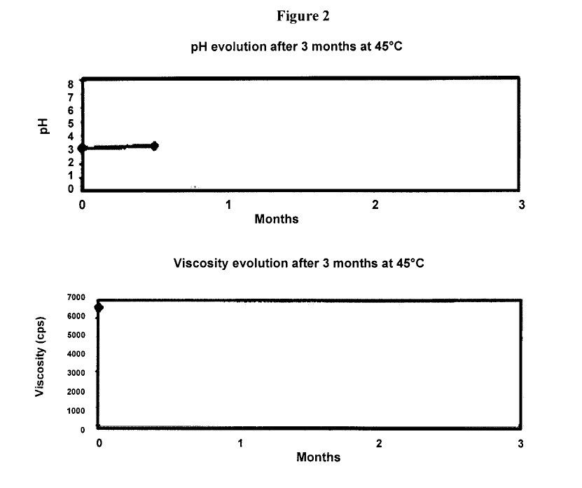

Formulation 2 was evaluated for its stability using four parameter measurements which included pH, texture, color and odour over a period of 3 months at 45° C. Formulation 2 was stable for less than 1 month whereby the formulation provided an average pH evolution of 3.195±0.09 with only one viscosity evolution reading of 6600 cps as illustrated in FIG. 2. Furthermore, the appearance of the cream produced beige and a brown dark color with notable surface oxidation. All measured parameters are illustrated in Table 4.

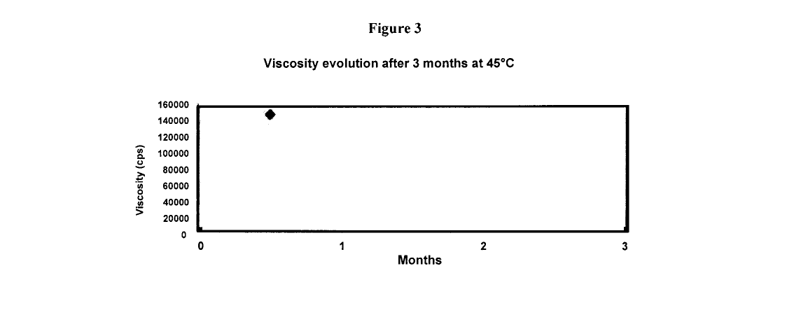

Similarly, another batch of formulation 2 was evaluated for its stability using four parameter measurements which included pH, texture, color and odour over a period of 3 months at 45° C. The second batch of formulation 2 was stable for less than 1 month whereby the formulation provided the same average pH evolution of 3.195±0.09 with only one viscosity evolution reading of 146000 cps as illustrated in FIG. 3. Furthermore, the appearance of the cream produced beige and a brown dark color with notable surface oxidation. All measured parameters are illustrated in Table 5.

### Storage Stability of Formulation 3.

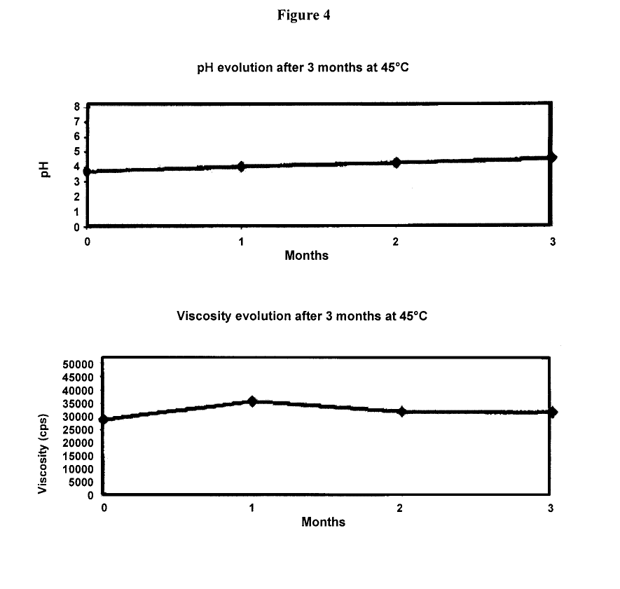

Formulation 3 was evaluated for its stability using four parameter measurements which included pH, texture, color and odour. Formulation 3 maintained its stability in all four parameters measured providing for an average pH evolution of 4.02±0.26 with a consistent viscosity evolution averaging at 31272 cps±2877 as depicted in both FIG. 4 and Table 7. However the color of Formulation 3 degraded to a dark brown within 2 months.

### Example 2: Doxycycline Stability Assessment in Exemplary Formulation 3

Preparation of Standard Series

2.0-5.0 mg of doxycycline hyclate, usp (Medisca, CAS 24390-14-5, code 0434-04, lot 59177/D) was weighed into a scintillation vial and the mass was recorded. The recorded mass was then used to calculate the volume of solvent needed to generate a final concentration of 1 mg/mL and that amount of 60:28:12 H₂O:CH₃CN:MeOH was accurately added to the vial using a pipette. The solution was vortexed for 30 seconds or until the doxycycline hyclate was completely dissolved.

A 50 μg/mL solution of doxycycline hyclate was prepared by adding 50 μL of 1 mg/mL stock solution to 950 μL of methanol/water (50:50) and the resulting solution was vortexed for 10 seconds. A 1 μg/mL solution doxycycline hyclate was prepared by adding 20 μL of 50 μg/mL solution to 980 μL of methanol/water (50:50) and the resulting solution was vortexed for 10 seconds. The 1 μg/mL doxycycline solution was serial diluted with methanol/water (50:50) to give concentrations of 1 μg/mL 500, 250, 125, 62.5, 31.25, 15.125, 7.8, and 3.9 ng/mL of doxycycline.

Peak area of doxycycline was plotted against doxycycline concentrations and used to produce a standard curve in the form of y=A+Bx using weighted least squares linear regression.

Control Samples

Preparation of Standards

Doped Cream—500 ng/mL

5-10 mgs of blank exemplary formulation 3 was weighed into a scintillation vial in duplicate. The mass was recorded and used to calculate the amount of 50 μg/mL solution of doxycycline to be added (mass of base formulation in mg % 100 gives μL of 50 μg/mL stock solution to add). Then enough methanol/water (50:50) was added to make up to 1 mg/mL (mass of base formulation in mg subtract mass of base formulation in mg % 100). The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 990 μL methanol/water (50:50) in a clean microcentrifuge tube and the solution was vortexed for 10 seconds. 100 μL of this solution was transferred by pipette to a HPLC vial with insert and capped.

Spiked Cream—13.5 μg/mL

5-10 mgs of blank exemplary formulation 3 was weighed into a scintillation vial in duplicate. The mass was recorded and used to calculate the amount of methanol/water (50:50) to add to make up to 1 mg/mL and this was added. The resulting solution was subjected to sonication for 30 minutes at room temperature. One mL of this solution was then transferred to a microcentrifuge tube and was centrifuged at 11000 rpm for 10 minutes. 10 μL of this solution was then added to 990 μL of a solution of 136 ng/mL of doxycycline in methanol/water (50:50) (made by adding 5.5 μL of 50 μg/mL to 1.9945 μL methanol/water (50:50) in a clean microcentrifuge tube and the solution was vortexed for 10 seconds. 100 μL of this solution was transferred by pipette to a HPLC vial with insert and capped.

Blank Samples

5-10 mgs of blank exemplary formulation 3 was weighed into a scintillation vial. The mass was recorded and used to calculate the amount of methanol/water (50:50) to add to make up to 1 mg/mL and this was added. The resulting solution was subjected to sonication for 30 minutes at room temperature. One mL of this solution was then transferred to a microcentrifuge tube and was centrifuged at 11000 rpm for 10 minutes. 10 μL of this solution was then added to 990 μL of a methanol/water (50:50) in a clean microcentrifuge tube and the solution was vortexed for 10 seconds. 100 μL of this solution was transferred by pipette to a HPLC vial with insert and capped.

Preparation of Unknown Samples

Unknown Samples

5-10 mgs of exemplary formulation 3 to be analyzed was weighed into a scintillation vial in duplicate. The mass was recorded and used to calculate the amount of methanol/water (50:50) to add to make up to 1 mg/mL and this was added. The resulting solution was subjected to sonication for 30 minutes at room temperature. One mL of this solution was then transferred to a microcentrifuge tube and was centrifuged at 11000 rpm for 10 minutes. 10 μL of this solution was then added to 990 μL of a methanol/water (50:50) in a clean microcentrifuge tube and the solution was vortexed for 10 seconds. 100 μL of this solution was transferred by pipette to an HPLC vial with insert and capped.

HPLC-UV Instrumentation and Conditions

The following methods were used for the LCMS analysis:

Isocratic chromatographic separation was performed on a C18 column (Zorbax eclipse XDB C18 column (4.6×150 mm, 5 micron particle size Agilent USKH009316) with guard using a mobile phase of MeOH (0.2% formic acid):water (0.4% formic acid) (60:40) at a flow rate of 0.75 mL/min for 5 min. The first two minutes was sent to the waste and doxycycline elutes between 3.5-4.5 mins. There post time was 0.1 min. The column temperature was 40° C. and the autosampler temperature was maintained at 4° C. The sample injection volume was 10 μL and the injector is set to −10 mm with bottom sensing enabled. A 4000 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 10, the ion spray voltage was 5000 volts, the temperature was 500° C., and gas sources 1 and 2 were 45 and 50 psi respectively. The declustering potential was 50 volts, the exit potential was 10.00 volts, the focusing lens 1 was −10.50 volts and the cell exit potential was 4.00 volts. Quantification was performed using the transitions m/z 444.8->428.2 (CE=28 V, 100 msec) for doxycycline with low resolution. Analytical data was acquired and quantification processing was performed by using Analyst software.

### Example 3: Clinical Formulation Efficacy of Doxycycline in Exemplary Formulations 2 and 3

164 patients participated in the clinical trial having ulcers, for example diabetic ulcers and ulcers as a result of other inflammatory, metabolic and/or autoimmune disorders, throughout various extremities of the body including leg, calf, shin, ankle, heel, stump, digits of the fingers and toes. The ulcers ranged in size from as small as 0.05 cm² to as large a surface area of 100 cm².

Generally, 0.5-1.0 g of cream was applied to the wound once per day, however the amount was varied depending on the size of the wound. The application was performed by a traveling nurse who applied the cream during the re-dressings of the wounds each day.

### Example 4: Doxycycline Stability Assessment Using Various Excipients in Exemplary Base Formulation 4 of the Application

Preparation of Formulations

Formulation 4:

Exemplary base formulation 4 (4 g) was added to a scintillation vial containing 1 mL of a penetration enhancer and the resulting suspension was vortexed on high for 10 minutes, sonicated for 30 seconds and then stirred on medium speed using the vortex for 2 hours. The formulation was then stirred with a spatula and evaluated visually for homogeneity.

Formulation 5:

Doxycycline hyclate (36.6 μmol/g) in exemplary base formulation 4.

Doxycycline hyclate (100 mgs), was weighed into a scintillation vial. 1 mL of a penetration enhancer (d=1.15 g/mL) was added to the vial and the solution was vortexed for 30 seconds until solubilized. A solution of 2 M NaOH was added dropwise until a pH of 5 to 6.5 was reached. The pH was recorded (5.5). 4 grams of exemplary base formulation 4 of the application was added to the vial and the resulting suspension was vortexed on high for 10 minutes, sonicated for 30 seconds and then stirred on medium speed using the vortexer for 2 hours. The formulation was subsequently stirred with a spatula and evaluated visually for homogeneity. (The final concentration of formulation 5 was adjusted for the density of the penetration enhancer, weight of excipients and addition of the NaOH_{aq} which was approximately 1.88% w/w).

Formulation 6:

Doxycycline hyclate (36.6 μmol/g)+MgCl₂ (36.6 μmol/g) in exemplary base formulation 4.

Doxycycline hyclate (100 mgs) and MgCl₂ (18.5 mgs) were weighed into a scintillation vial. 1 mL of a penetration enhancer was added to the vial and the solution was vortexed for 30 seconds until solubilized. A solution of 2 M NaOH was added dropwise until a pH of 5 to 6.5 was reached. The pH was recorded (6.5). 4 grams of exemplary base formulation 4 was added to the vial and the resulting suspension was vortexed on high for 10 minutes, sonicated for 30 seconds and then stirred on medium speed using the vortex for 2 hours. The formulation was subsequently stirred with a spatula and evaluated visually for homogeneity. (The final concentration of formulation 6 was adjusted for the density of the penetration enhancer, weight of excipients and addition of the NaOH_{aq} which was approximately 1.88% w/w).

Formulation 7:

Doxycycline hyclate (35.8 μmol/g)+MgCl₂ (35.8 μmol/g)+2-hydroxypropyl-(β-cyclodextrin (35.8 μmol/g) in exemplary base formulation 4.

Doxycycline hyclate (100 mgs), MgCl₂ (18.5 mgs) and 2-hydroxypropyl-β-cyclodextrin (268 mgs) were weighed into a scintillation vial. 1 mL of distilled water was added to the vial and the solution was vortexed for 30 seconds until solubilized. A solution of 2 M NaOH was added dropwise until a pH of 5 to 6.5 was reached. The pH was recorded (5.6). 4 grams of exemplary base formulation 4 was added to the vial and the resulting suspension was vortexed on high for 10 minutes, sonicated for 30 seconds and then stirred on medium speed using the vortex for 2 hours. The formulation was then stirred with a spatula and evaluated visually for homogeneity. (The final concentration of formulation 7 was adjusted for the weight of excipients and addition of the NaOH_{aq} which was approximately 1.84% w/w).

Formulation 8:

Doxycycline hyclate (36.0 μmol/g)+2-hydroxypropyl-β-cyclodextrin (36.0 μmol/g) in exemplary base formulation 4.

Doxycycline hyclate (100 mgs) and 2-hydroxypropyl-β-cyclodextrin (268 mgs) were weighed into a scintillation vial. 1 mL of distilled water was added to the vial and the solution was vortexed for 30 seconds until solubilized. A solution of 2 M NaOH was added dropwise until a pH of 5 to 6.5 was reached (5 drops˜50 μL). The pH was recorded. (5.9) 4 grams of exemplary base formulation 4 was added to the vial and the resulting suspension was vortexed on high for 10 minutes, sonicated for 30 seconds and then stirred on medium speed using the vortex for 2 hours. The formulation was then stirred with a spatula and evaluated visually for homogeneity. ((The final concentration of formulation 8 was adjusted for the weight of excipients and addition of the NaOH_{aq} which was approximately 1.85% w/w).

Storage

Sample portions of each formulation were transferred to two additional containers (eppendorf tubes). The samples were then stored at −20° C., 5° C. and 25° C. in the dark.

The concentration of doxycycline in each cream was quantified on day 0 and were within acceptable ranges. The samples stored at −20° C. were used as the tₒ concentrations and the concentration of doxycycline stored at 5° C. and 25° C. are described as % reduction of doxycycline.

Quantification of Doxycycline in Cream Formulations

Preparation of Standard Series:

2.0-5.0 mg of doxycycline hyclate, usp (Medisca, CAS 24390-14-5, code 0434-04, lot 59177/D) was weighed into a scintillation vial and the mass was recorded. The recorded mass was then used to calculate the volume of solvent needed to generate a final concentration of 1 mg/mL and that amount of 60:28:12 H₂O:ACN:MeOH was accurately added to the vial using a pipette. The solution was vortexed for 30 seconds or until the doxycycline hyclate was completely dissolved.

A 50 μg/mL solution of doxycycline hyclate was prepared by adding 50 μL of 1 mg/mL stock solution to 950 μL of methanol/water (50:50) and the resulting solution was vortexed for 10 seconds. A 1 μg/mL solution doxycycline hyclate+125 ng/mL d3-doxycycline was prepared by adding 40 μL of 50 μg/mL solution doxycycline hyclate and 12.5 μL of a 20 μg/mL solution of d3-doxycycline to 1.9475 μL of methanol/water (50:50) and the resulting solution was vortexed for 10 seconds (A). The 125 ng/mL solution d3-doxycycline was prepared by adding 12.5 μL of 20 μg/mL d3-doxycycline solution to 1.9875 μL of 50:50 methanol/water (B). Solution A was serial diluted with solution B to give a standard series with concentrations of 1 μg/mL 500, 250, 125, 62.5, 31.25, 15.125, 7.8, and 3.9 ng/mL of doxycycline and a constant concentration of 125 ng/mL of d3-doxycycline.

Ratio of peak area of doxycycline to peak area of d3-doxycyline was plotted against doxycycline concentrations and used to produce a standard curve in the form of y=A+Bx using weighted least squares linear regression.

Quality Control Samples:

Spiked Cream Doxycycline Hyclate (1.72% Doxycycline):

5-10 mgs of exemplary base formulation 4 was weighed into a scintillation vial in duplicate. The mass was recorded and used to calculate the amount of methanol/water (50:50 adjusted to pH 2 with 1 M HCl) to add to make a 1 mg/mL solution and that was added by pipette. The resulting solution was subjected to sonication for 30 minutes at room temperature. 1 mL of this solution was then transferred to a microcentrifuge tube by pipette and was centrifuged at 11000 rpm for 10 minutes. 10 μL of this solution was then added to 990 μL of a 202 ng/mL doxycycline+125.6 ng/ml d3-doxycycline. (made by adding 25.12 μL of 20 μg/mL solution of d3-doxycycline and 16.16 μL of a 50 μg/mL of doxycycline to 3.959 mL of 50:50 methanol:water) in a microcentrifuge tube and vortex. 100 μL of this solution was then transferred to a HPLC vial with insert.

Blank Cream:

5-10 mgs of exemplary base formulation 4 was weighed into a scintillation vial in duplicate. The mass was recorded and used to calculate the amount of methanol/water (50:50 adjusted to pH 2 with 1 M HCl) to add to make a 1 mg/mL solution and that was added by pipette. The resulting solution was subjected to sonication for 30 minutes at room temperature. 1 mL of this solution was then transferred to a microcentrifuge tube by pipette and was centrifuged at 11000 rpm for 10 minutes. 10 μL of this solution was then added to 990 μL of a 125.6 ng/mL solution of d3-doxycycline hyclate in methanol/water (50:50) in a microcentrifuge tube (made by adding 125.6 μL of 50 μg/mL solution to 19.9875 mL methanol/water (50:50)) vortexed for 15 seconds and 100 μL of the resulting solution in a HPLC vial with insert.

Doped Cream:

5-10 mgs of exemplary base formulation 4 was weighed into a scintillation vial in duplicate. The mass was recorded and used to calculate the amount 1 mg/mL solution of doxycycline hyclate to be added to the dope (0.02*mass in mgs=volume in μL). Methanol/water (50:50 adjusted to pH 2 with 1 M HCl) was then added to make a 1 mg/mL solution by pipette. The resulting solution was subjected to sonication for 30 minutes at room temperature. 1 mL of this solution was then transferred to a microcentrifuge tube by pipette and was centrifuged at 11000 rpm for 10 minutes. 10 μL of this solution was then added to 990 μL of a 125.6 ng/mL solution of d3-doxycycline hyclate in methanol/water (50:50) in a microcentrifuge tube (made by adding 125.6 μL of 50 μg/mL solution to 19.9875 mL methanol/water (50:50)) vortexed for 15 seconds and 100 μL of the resulting solution in a HPLC vial with insert.

Sample Preparation:

5-10 mgs of the formulation to be tested was weighed into a scintillation vial in duplicate. The mass was recorded and used to calculate the amount of extraction solvent (50:50 MeOH:H₂O adjusted to pH 2 with 1 M HCl) to add to make a 1 mg/mL solution and that was added by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 990 μL of a 125.6 ng/mL solution of d3-doxycycline hyclate in methanol/water (50:50) in a microcentrifuge tube (made by adding 125.6 μL of 50 μg/mL solution to 19.9875 mL methanol/water (50:50)) vortexed for 15 seconds and 100 μL of the resulting solution in a HPLC vial with insert.

HPLC-UV Instrumentation and Conditions:

Isocratic chromatographic separation was performed on a C18 column (Zorbax eclipse XDB C18 column (4.6×150 mm, 5 micron particle size Agilent USKH009316) with guard using a mobile phase of ACN (0.1% trifluoroacetic acid):water (0.1% trifluoroacetic acid) (70:30) at a flow rate of 0.75 mL/min for 10 min. The first two minutes was sent to the waste and doxycycline elutes between 7-8 mins. There was no post time. The column temperature was 40° C. and the autosampler temperature was maintained at 4° C. The sample injection volume was 10 μL and the injector is set to −10 mm with bottom sensing enabled. A 4000 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 10, the ion spray voltage was 5000 volts, the temperature was 500° C., and gas sources 1 and 2 were 45 and 50 psi respectively. The declustering potential was 50 volts, the exit potential was 10.00 volts, the focusing lens 1 was −10.50 volts and the cell exit potential was 4.00 volts. Quantification was performed using the transitions m/z 444.8->428.2 (CE=28 V, 100-msec) for doxycycline and 447.8->431.2 (CE=28 V, 100 msec) for d3-doxycycline with low resolution. Analytical data was acquired and quantification processing was performed by using Analyst software.

### IV. Results and Discussion

### (1) Stability: Quantification of Doxycycline in Exemplary Formulations 1-3 Base Creams

A study was conducted to extract and quantify the doxycycline in exemplary formulation 3 to establish the stability and consistency of doxycyclin in creams. Preliminary clinical evidence for doxycycline in exemplary formulation 1-3 indicates that these formulations produce better wound healing outcomes than standard topical formulations of doxycycline, including, for example doxycycline formulated in Glaxal™ base and Pluronic lecithin organogel (PLO, Murdan, Sudaxshina in Hospital Pharmacist, July/August 2005, Vol. 12, pp/267-270). These formulations will color over time. Therefore, a methodology was developed to extract and quantify the doxycycline from the exemplary formulation 3 to provide further insight in improving overall stability.

The purpose of this study was to optimize the stability of doxycycline within the exemplary formulations of the application and fully characterize its thermostability and degradation in the product formulations over time.

Doxycycline is often packaged as a lyophilized powder, and is reported to be unstable in compounded formulations and solution. The first aim was to design formulations with an optimized doxycycline stability profile. The three formulations were prepared and 3 samples of each cream formulations were reserved for analysis.

Doxycycline was extracted in methanol:water from cream formulations by sonication, diluted and then quantified using liquid chromatography-triple quadruple mass spectrometry (LC-MS/MS). A calibration curve calculated by the peak area ratio of concentration of an analytical standard of doxycycline to an internal standard was used to calculate the concentrations of unknowns and establish linearity and LLOQ. Standards were included to confirm assay specificity (blanks), recovery (dopes) and precision and accuracy (spikes). All methods were performed in conformance with the U.S. FDA guidance document for Industry

The linear range of quantification for this methodology was 3.9 ng/mL to 1000 ng/mL with a limit of detection and limit of quantification of <3.9 ng/mL. Control concentrations of doxycycline were within acceptable ranges with spiked doxycycline concentration of 101.9% for cream samples indicating matrix effects are negligible between standards and unknown samples (data not shown). Control concentrations of doped doxycycline demonstrated an extraction efficiency of 108.3% from cream samples indicating an acceptable extraction procedure (data not shown).

Doxycycline formulations 1-3 were included in the study. The three creams were formulations prepared in May, June and July of 2015 and stored between 5-25° C. until analysis in July of 2015.

The three formulations tested: Formulation 1, Formulation 2 and Formulation 3 had average concentrations of 2.18+/−0.01% w/w (mean+/−SEM) (formulated in July, 2015), 2.30+/−0.10% w/w (mean+/−SEM) (formulated in June, 2015), and 2.15+/−0.01% w/w (mean+/−SEM) (formulated in May, 2015), respectively.

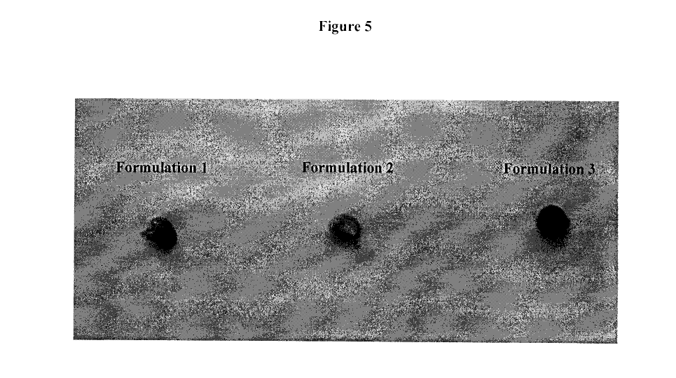

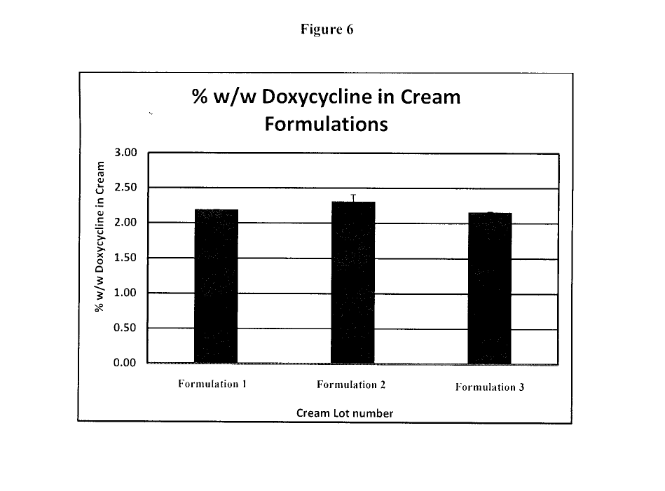

There was a dramatic color change in formulation 3 (oldest, May) to dark brown (FIG. 5). The observed visual change does not appear to correspond to significant change in concentration in doxycycline however, as the concentrations of doxycycline within all three formulations were all determined to be within 2.15-20.3%, suggesting the formulation concentration remains within 7.5% of the original concentration over the period of three months (FIG. 6) under the conditions tested.

The data supports the hypothesis that there is no significant degradation (<10%) of the doxycycline over the time period studied under the storage conditions and the observed color change was not the result of a major reduction in the concentration of doxycycline.

### (2) Clinical Formulation Efficacy of Doxycycline in Exemplary Formulations 2 and 3 Base Creams

The primary objective of the study was to assess the efficacy of doxycycline in exemplary formulations 2 and 3 of the present application to yield ulcer closure (re-epithelialization) from baseline to week 12.

164 patients participated in the clinical trial having ulcers throughout various extremities of the body including leg, calf, shin, ankle, heel, stump, digits of the fingers and toes. The ulcers ranged in sizes from as small as 0.05 cm² to as large a surface area of 100 cm². 0.5-1.0 g of the doxycycline cream was applied to the wound of each patient once per day, however the amount of cream applied was dependent on the size of the wound.

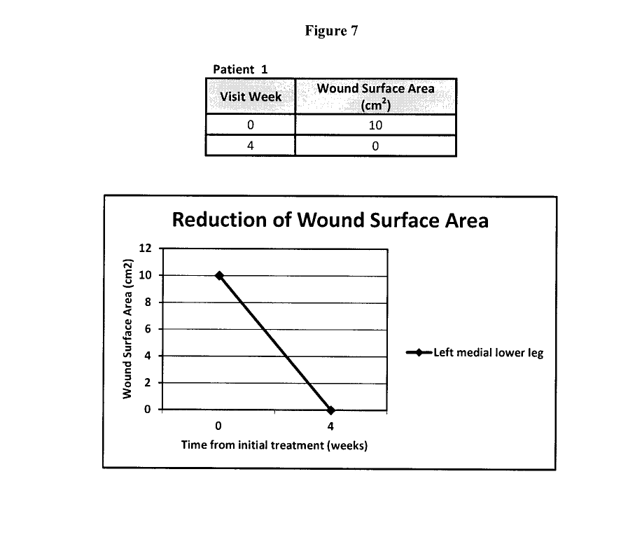

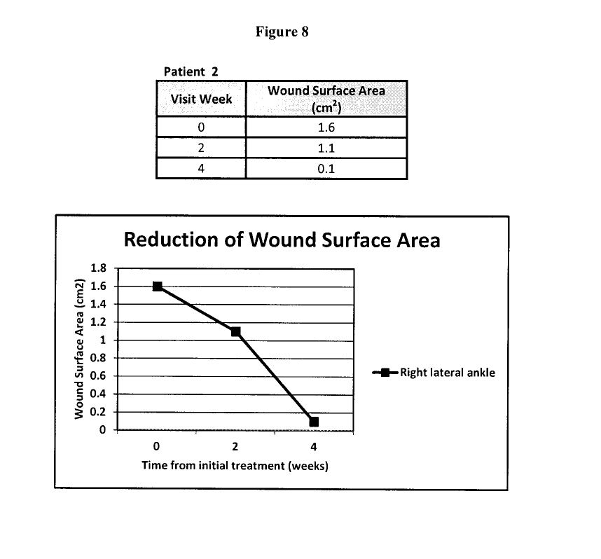

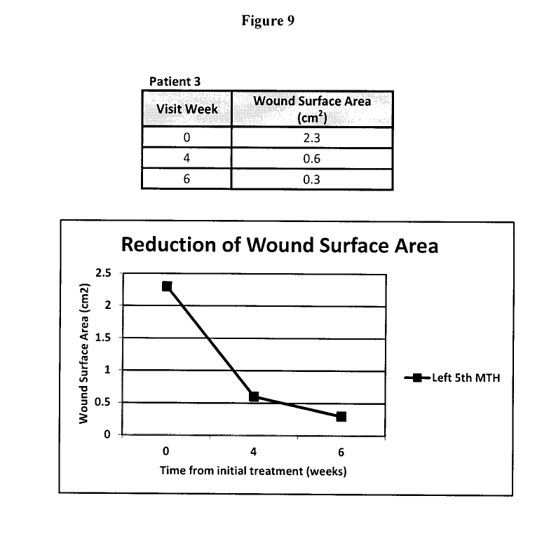

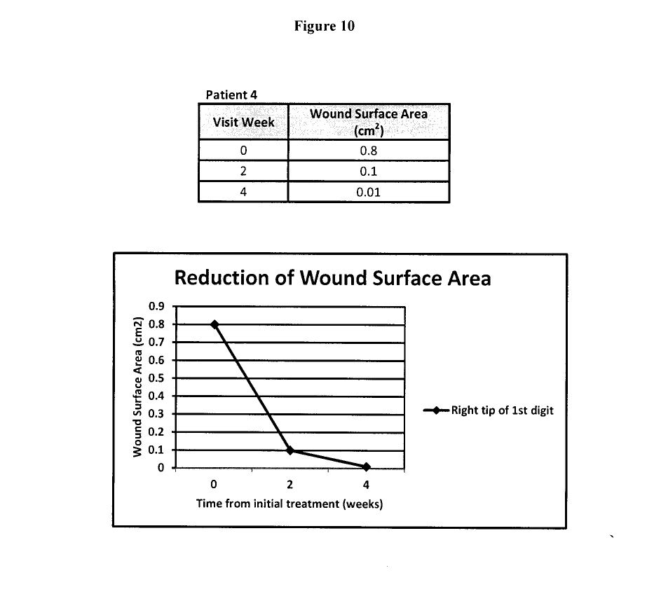

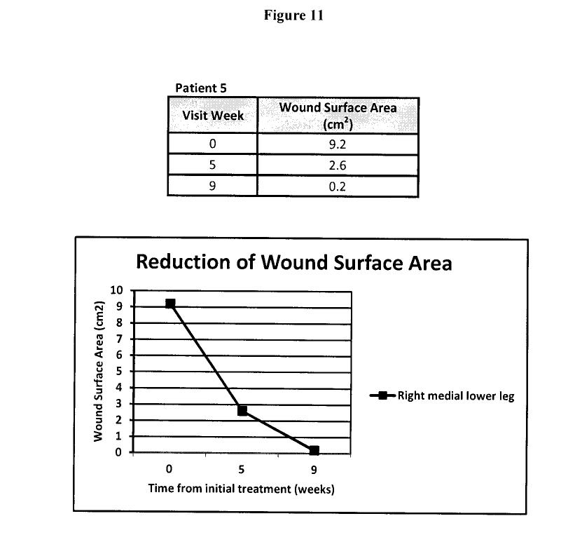

56 of the 164 patients (˜34%) had complete healing of their ulcer wound. 60 patients (˜36%) had varying degrees of improvement in the re-epithelialization of their ulcer wound as illustrated on tables 8 and 9. On the other hand, 48 patients (˜29%) were observed to have no improvement. Generally, the doxycycline active within the exemplary transdermal base formulations 2 and 3 were able to induce re-epithelialization within 2 weeks of treatment, with results of complete cessation of the ulcer wound observed as early as 6 weeks of treatment for a majority of the participants (94.1%, Table 10, FIGS. 7-11).

### (3) Doxycycline Stability Assessment Using Various Excipients in Exemplary Base Formulation 4 of the Application

Exemplary formulations 1-3 appear to change color over time, and succumb to the common reported issue of the poor stability of doxycycline. As such, the primary objective of this study was on the development of a methodology to extract and quantify the doxycycline from exemplary base formulation 4.

The study included the analysis of several variations of the formulations of doxycycline-exemplary base formulation 4 to determine the best formulation conditions to optimize for the stability of doxycycline. The goal of the study was to produce a formulation of doxycycline that is stable (<+/−5% doxycycline) over a period of three months.

The standard series shows good inter-day reproducibility and the concentrations 3.9 ng/mL-1 μg/mL fall within the linear range of doxycycline. Control concentrations of doped doxycycline demonstrated an extraction efficiency of 111.6%+/−11 (mean+/−SEM) and 104.4%+/−2 (mean+/−SEM) for spiked samples indicating an acceptable extraction procedure and minimal matrix effects. This method had a limit of detection (LOD)<3.9 ng/mL.

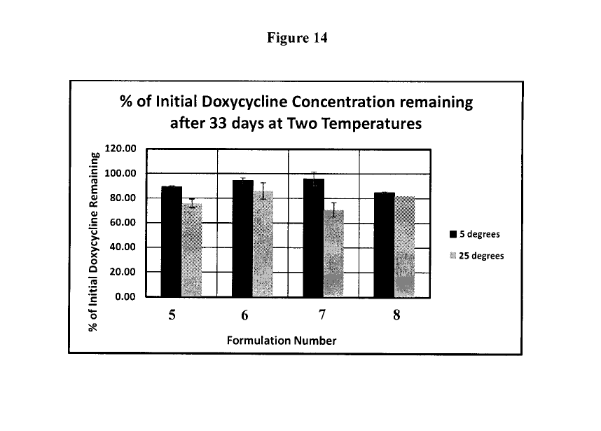

FIG. 14 illustrates the percentage of initial doxycycline concentration remaining in exemplary base formulation 4 after 33 days compared. Formulation 5: Doxycycline hyclate (36.6 μmol/g) in exemplary base formulation 4 shows 89.1%+/−0.7 (Average+/−SD) of the initial doxycycline concentration left after 33 days of storage at 5° C. and 75.6+/−3.4 (Average+/−SD) at 25° C. Formulation 6: Doxycycline hyclate (36.6 μmol/g)+MgCl₂ (36.6 μmol/g) in exemplary base formulation 4, shows 94.2%+/−2.3 (Average+/−SD) of the initial doxycycline concentration left after 33 days of storage at 5° C. and 85.9+/−6.6 (Average+/−SD) at 25° C. Formulation 7: Doxycycline hyclate (35.8 μmol/g)+MgCl₂ (35.8 μmol/g)+2-hydroxypropyl-β-cyclodextrin (35.8 μmol/g) in exemplary base formulation 4 shows 95.9%+/−5.6 (Average+/−SD) of the initial doxycycline concentration left after 33 days of storage at 5° C. and 70.8+/−5.6 (Average+/−SD) at 25° C. Formulation 8: Doxycycline hyclate (36.0 μmol/g)+2-hydroxypropyl-β-cyclodextrin (36.0 μmol/g) in exemplary base formulation 4 contains 84.8%+/−0.6 (Average+/−SD) of the initial doxycycline concentration left after 33 days of storage at 5° C. and 82.1 (no replicates to facilitate a standard deviation) at 25° C.

The storage of the doxycycline-exemplary base formulation 4 samples at 5° C. are universally better for the stability of the doxycycline in the current formulations than storage at room temperature. The presence of magnesium alone or magnesium with β-cyclodextrin appears to further stabilize the doxycycline at 5° C. compared to the doxycycline control or doxycycline with β-cyclodextrin. At room temperature, the doxycycline and magnesium (formulation 6) appears to be the most stable and the second most stabilized formulation contained β-cyclodextrin only.

The goal of generating a set of conditions where the doxycycline is stable (+/−5%) over 30 days was successfully completed in formulation 7 stored at 5° C.

### Example 5: Stability of Doxycycline in Base Formulation

Preparation of Standard Series

2.0-5.0 mg of doxycycline hyclate, usp (Medisca, CAS 24390-14-5, code 0434-04, lot 59177/D) was weighed into a scintillation vial and the mass was recorded. The recorded mass was then used to calculate the volume of solvent needed to generate a final concentration of 1 mg/mL and that amount of 60:28:12 H₂O:ACN:MeOH was accurately added to the vial using a pipette. The solution was vortexed for 30 seconds or until the doxycycline hyclate was completely dissolved.

A 50 μg/mL solution of doxycycline hyclate was prepared by adding 50 μL of 1 mg/mL stock solution to 950 μL of methanol/water (50:50) and the resulting solution was vortexed for 10 seconds. A 1 μg/mL solution doxycycline hyclate+125 ng/mL d3-doxycycline was prepared by adding 40 μL of 50 μg/mL solution doxycycline hyclate and 12.5 μL of a 20 μg/mL solution of d3-doxycycline to 1.9475 μL of methanol/water (50:50) and the resulting solution was vortexed for 10 seconds (A). The 125 ng/mL solution d3-doxycycline was prepared by adding 12.5 μL of 20 μg/mL d3-doxycycline solution to 1.9875 μL of 50:50 methanol/water (B). Solution A was serial diluted with solution B to give a standard series with concentrations of 1000, 500, 250, 125, 62.5, 31.25, 15.125, 7.8, and 3.9 ng/mL of doxycycline and a constant concentration of 125 ng/mL of d3-doxycycline.

Ratio of peak area of doxycycline to peak area of d3-doxycyline was plotted against doxycycline concentrations and used to produce a standard curve in the form of y=A+Bx using weighted least squares linear regression.

Quality Control Samples:

Doped Cream:

5-10 mgs of blank base cream prepared according to Examples 1 and 4 (Table 12; Formulation 9) was weighed into a scintillation vial in triplicate. The mass was recorded and used to calculate the amount 1 mg/mL solution of doxycycline hyclate to be added to the dope (0.02*mass in mgs=volume in μL). Enough methanol/water (50:50 adjusted to pH 2 with 1 M HCl) was then added to make a 1 mg/mL solution by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 990 μL of a 125.6 ng/mL solution of d₃-doxycycline hyclate in methanol/water (50:50) in a microcentrifuge tube (made by adding 125.6 μL of 50 μg/mL solution to 19.9875 mL methanol/water (50:50)) vortexed for 15 seconds and 100 μL of the resulting solution was transferred to an HPLC vial with insert.

Sample Preparation:

5-10 mgs of cream to be tested was weighed into a scintillation vial in duplicate. The mass was recorded and used to calculate the amount of extraction solvent (50:50 MeOH:H₂O adjusted to pH 2 with 1 M HCl) to add to make a 1 mg/mL solution and that was added by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 990 μL of a 125.6 ng/mL solution of d₃-doxycycline hyclate in methanol/water (50:50) in a microcentrifuge tube (made by adding 125.6 μL of 50 μg/mL solution to 19.9875 mL methanol/water (50:50)) vortexed for 15 seconds and 100 μL of the resulting solution was transferred to an HPLC vial with insert.

HPLC-MS Instrumentation and Conditions:

Isocratic chromatographic separation was performed on a C18 column (Eclipse XDB C18 column (4.6×150 mm, 5 micron particle size Agilent USKH095544) with guard using a mobile phase of ACN (0.1% trifluoroacetic acid):water (0.1% trifluoroacetic acid) (30:70) at a flow rate of 0.75 mL/min for 10 min. The first two minutes was sent to the waste and doxycycline elutes between 6.5-7.5 mins. There was no post time. The column temperature was 30° C. and the autosampler temperature was maintained at 4° C. The sample injection volume was 5 μL and the injector is set to 0 mm with bottom sensing enabled. A 5500 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was medium, the ion spray voltage was 5000 volts, the temperature was 500° C., and gas sources 1 and 2 were 45 and 50 psi respectively. The declustering potential was 40 volts, the exit potential was 10.00 volts, the focusing lens 1 was −10.50 volts and the cell exit potential was 4.00 volts. Quantification was performed using the transitions m/z 444.8->428.2 (CE=28 V, 100-msec) for doxycycline and 447.8->431.2 (CE=28 V, 100 msec) for d₃-doxycycline with low resolution. Analytical data was acquired and quantification processing was performed by using Analyst software.

Results and Discussion

The linear range for doxycycline hyclate using this method is 3.9 ng/mL-1000 ng/mL. LLOD for (3× average blank peak area) and LLOQ (10× average blank peak area) for doxycycline hyclate using this method are both <3.9 ng/mL. Control doped samples had extraction efficiency of 107.1%+/−1.1 (% recovered+/−SEM) indicating an acceptable extraction procedure.

The cream was tested for doxycycline hyclate concentration day 1, 14 days later, and 13 days after that. The cream did appear to have a slight decline in the doxycycline concentration, however after 27 days, the concentration was within 20% of the original concentration. Real time stability testing of doxycycline hyclate concentrations within the formulation determined that the doxycycline hyclate concentration started around 2.7% w/w and declined slightly over 27 days, however remained within 20% of the original concentration under the storage conditions. No overt decomposition was identified.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Formulation 1

| | Ingredients | % |
|---|---|---|
| Phase A | Emulsifier | 4.0% |
| | Wax Stabilizer | 3.0% |
| | Wax Emollient | 1.00% |
| | Polar Emollient Oils | 5-9% |
| | Medium Polar Emollient | 3% |
| Phase B | Water | 49.4% |
| | Arginine | 0.60% |
| | Thickening agent | 0.50% |
| | Humectant | 4% |
| Phase C | Preservative | 1% |
| Phase D | Flavonoid-containing | 1-3% |
| | extracts | |
| | Penetration Enhancer | 3.0-4.5% |
| | Thickening Agent | 1.0% |
| | Water | 6.0% |
| | Antioxidant-Ascorbyl | 0.10% |
| | Ester | |
| | Phospholipid- | 2.0% |
| | Complexed Flavonoid | |
| Phase E | Doxycyclin Hyclate | 2.0% |
| | Water | 5.9% |
| | Total | 100.00% |

TABLE 2
Stability parameters, pH, texture, color and odour for formulation 1 at
45° C. for 90 days

| | | | Viscosity | | | |
|---|---|---|---|---|---|---|
| | Avg. | | (T4- | | | |
| | Temp | | 100 rpm, | | | |
| Months | (° C.) | pH | cps) | Appearance | Colour | Scent |
| 0 | 25 | 3.16 | 2556 | cream | beige | characteristic |
| 0.5 | 25 | 3.16 | 4374 | cream + | beige | characteristic |
| | | | | surface | | |
| | | | | oxidation | | |
| 1 | 25 | 3.25 | 5466 | cream + | beige | characteristic |
| | | | | surface | | |
| | | | | oxidation | | |
| 2 | 25 | | | | | |
| 3 | 25 | | | | | |

TABLE 3
Formulation 2

| | Ingredients | % |
|---|---|---|
| Phase A | Emulsifier | 4.0% |
| | Wax Stabilizer | 3.0% |
| | Wax Emollient | 1.00% |
| | Polar Emollient Oils | 5-9% |
| | Medium Polar Emollient | 3% |
| Phase B | Water | 49.4% |
| | Arginine | 0.60% |
| | Thickening agent | 0.50% |
| | Humectant | 4% |
| Phase C | Preservative | 1% |
| Phase D | Flavonoid-containing | 1-3% |
| | extracts | |
| | Penetration Enhancer | 3.0-4.5% |
| | Thickening Agent | 1.0% |
| | Water | 2.0% |
| | Antioxidant-Rosemary | 0.50% |
| | extract | |
| | Phospholipid- | 2.0% |
| | Complexed Flavonoid | |
| Phase E | Doxycyclin Hyclate | 2.0% |
| | Water | 9.5% |
| | Total | 100.00% |

TABLE 4
Stability parameters, pH, texture, color and odour for formulation 2 at
45° C. for 90 days

| | | | Viscosity | | | |
|---|---|---|---|---|---|---|
| | Avg. | | (T3- | | | |
| | Temp | | 10 rpm, | | | |
| Months | (° C.) | pH | cps) | Appearance | Colour | Scent |
| 0 | 25 | 3.13 | 6600 | cream | beige | characteristic |
| 0.5 | 25 | 3.26 | | cream + | brown | characteristic |
| | | | | surface | dark | |
| | | | | oxidation | | |
| 1 | 25 | | | | | |
| 2 | 25 | | | | | |
| 3 | 25 | | | | | |

TABLE 5
Stability parameters, pH, texture, color and odour for formulation 2 at
45° C. for 90 days

| | | | Viscosity | | | |
|---|---|---|---|---|---|---|
| | Avg. | | (T3- | | | |
| | Temp | | 03 rpm, | | | |
| Months | (° C.) | pH | cps) | Appearance | Colour | Scent |
| 0 | 25 | 3.13 | | cream | beige | characteristic |
| 0.5 | 25 | 3.26 | 146000 | cream + | brown | characteristic |
| | | | | surface | dark | |
| | | | | oxidation | | |
| 1 | 25 | | | | | |
| 2 | 25 | | | | | |
| 3 | 25 | | | | | |

TABLE 6
Formulation 3

| | Ingredients | % |
|---|---|---|
| Phase A | Emulsifier | 4.0% |
| | Wax Stabilizer | 3.0% |
| | Wax Emollient | 1.00% |
| | Polar Emollient Oils | 5-9% |
| | Medium Polar Emollient | 3% |
| Phase B | Water | 49.4% |
| | Arginine | 0.60% |
| | Thickening agent | 0.50% |
| | Humectant | 4% |
| Phase C | Preservative | 1% |
| Phase D | Flavonoid-containing | 1-3% |
| | extracts | |
| | Penetration Enhancer | 3.0-4.5% |
| | Thickening Agent | 1.0% |
| | Water | 2.0% |
| | Phospholipid- | 2.0% |
| | Complexed Flavonoid | |
| Phase E | Doxycyclin Hyclate | 2.0% |
| | Water | 10% |
| | Total | 100.00% |

TABLE 7
The stability parameters, pH, texture, color and odour for formulation
3 at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 3.69 | 28860 | cream | beige- | characteristic |
| | | | | | yellow | |
| 1 | 25 | 3.97 | 35380 | cream + | greenish | characteristic |
| | | | | surface | beige | |
| | | | | oxidation | | |
| 2 | 25 | 4.14 | 31020 | cream + oil + | brown dark | characteristic |
| | | | | surface | | |
| | | | | oxidation | | |
| 3 | 25 | 4.30 | 29830 | cream + oil + | brown dark | characteristic |
| | | | | surface | | |
| | | | | oxidation | | |

TABLE 8

| SUMMARY | TOTAL # | % |
|---|---|---|
| Healed Ulcers | 56 | 34.1463415 |
| No improvement | 48 | 29.2682927 |
| Less than 25% | 6 | 3.65853659 |
| decrease | | |
| More than 25% | 25 | 15.2439024 |
| decrease but less | | |
| than 50% | | |
| More than 50% | 12 | 7.31707317 |
| but less than | | |
| 75% decrease | | |
| More than 75% | 17 | 10.3658537 |
| and less than | | |
| 100% | | |

TABLE 9

| | TOTAL # | % |
|---|---|---|
| Healed ulcers | 56 | 34.14634 |
| No | 48 | 29.26829 |
| improvement | | |
| Improvement | 60 | 36.58537 |

TABLE 10

| % of ulcers | |
|---|---|
| healed at # | |
| weeks | |
| 2 w | 17.3 |
| 3 w | 25 |
| 4 w | 26.9 |
| 5 w | 9.6 |
| 6 w | 15.3 |
| 8 w | 1.9 |
| 9 w | 1.9 |
| 10 w | 1.9 |
| 12 w | 1.9 |

TABLE 11
Formulation 4

| | Ingredients | % |
|---|---|---|
| Phase A | Emulsifier | 4.0% |
| | Wax Stabilizer | 3.0% |
| | Wax Emollient | 2.0% |
| | Polar Emollient Oils | 10.0% |
| | Medium Polar Emollient | 3.0% |
| Phase B | Water | 58.70% |
| | Thickening agent | 0.5% |
| | Humectant | 4.0% |
| Phase C | Preservatives | 1.3% |
| Phase D | Flavonoid-containing | 7.0% |
| | extracts | |
| | Penetration Enhancer | 1.5% |
| | Thickening Agent | 1.0% |
| | Water | 2.0% |
| | Phospholipid- | 2.0% |
| | Complexed Flavonoid | |
| | Total | 100.00% |

TABLE 12
Formulation 9

| | Ingredients | % |
|---|---|---|
| Phase A | Emulsifier | 4.5% |
| | Wax Stabilizer | 3.5% |
| | Polar Emollient Oils | 9.0% |
| | Medium Polar Emollient | 3.0% |
| Phase B | Water | 30.70% |
| | Arginine | 0.60% |
| | Thickening agent | 0.5% |
| | Humectant | 4.0% |
| Phase C | pH Adjuster | 0.7% |
| | Preservatives | 1.0% |
| Phase D | Thickening Agent | 1.0% |
| Phase E | Flavonoid-containing | 3.0% |
| | extracts | |
| | Penetration Enhancer | 4.5% |
| | Thickening Agent | 1.0% |
| | Preservative/Emollient | 0.50% |
| | Water | 5.0% |
| | Phospholipid- | 2.0% |
| | Complexed Flavonoid | |
| Phase F | Water | 17.50% |
| | Cyclodextrine | 4.00% |
| | Essential Minerals | 3.00% |
| | Doxycycline Hyclate | 2.00% |
| | Total | 100.00% |

### REFERENCES

1. Ajabnoor, M. A. (1990). Effects of Aloes on blood glucose levels in normal and alloxan diabetic mice. J. Ethnophrmacol. 28: 215-220.
2. Agren, M. S., Mirastschijski, U, Karlsmark, T., Saarialho-Kere, U. K. (2001). Topical synthetic inhibitor of matrix metalloproteinases delays epidermal regeneration of human wounds. Exp Dermatol. 10: 337-348.
3. Brem, H., Tomic-Canic, M. (2007). Cellular and molecular basis of wound healing in diabetes. J Clin Invest 2007, 117, 1219-1222.
4. Chin, G. A., Thigpin, T. G., Perrin, K. J., Moldawer, μL. μL., Schultz, G. S. (2003). Treatment of chronic ulcers in diabetic patients with a topical metalloproteinase inhibitor, doxycycline. Wounds. 15(10).
5. Collins, N. Advances in skin and wound care. The Journal for Prevention and Healing 14(1):16-17.
6. Diegelmann, R. F., m Evans, M. C. (2004). Wound healing: an overview of acute, fibrotic and delayed healing. Frontiers in Bioscience, 9, 283-289.
7. Falanga, V. (2005). Wound healing and its impairment in the diabetic foot. The Lancet. Vol 366, No. 9498. 1736-1743.
8. Falanga, V., Saap, μL. J., Ozonoff, A. (2006). Wound bed score and its correlation with healing of chronic wounds. Dermatol Ther. 19(6):383-390.
9. Galiano, R. D., Michaels, J. 5^{th}, Dobryansky, M., Levine, J. P., Gurtner, G. C. (2004). Quantitative and reproducible murine model of excisional wound healing. Wound repair Regen 12: 485-492.
10. Golub, μL. M., Ramamurthy, N. S., McNamara, T. F., Greenwald, R. A., Rifkin, B. R. (1991). Tetracyclines inhibit connective tissue breakdown: new therapeutic implications for an old family of drugs. Crit Rev Oral Biol Med. 2(3): 297-321.
11. Greenland, R. A. (1994). Treatment of destructive arthritic disorders with MMP inhibitors. Potential role for tetracyclines. Ann N Y Acad Sci. 732: 181-198.
12. James, R. W., Scott, C. (2012). Doxycycline as a modulator of inflammation in chronic wounds. Wounds 24 (12): 339-349.
13. Joyce, S., Linda, c., Gregory, S. (2010). The role off doxycycline as a matrix metalloproteinase inhibitor for the treatment of chronic wounds. Biol. Res. Nurs. 11: 336-344.
14. Kirchner, μL. M., Meerbaum, S. O., Gruber, B. S (2003). Effects of vascular endothelial growth factor on wound closure rates in the genetically diabetic mouse model. Wound Repair Regen. 11:127-131.
15. Lee, H. J., Yang, S. H., Oh, J. M., Lee, M. G. (2010). Pharmacokinetics of drugs in rats with diabetes mellitus induced by alloxan or streptozocin: comparison with those in patients with type 1 diabetes mellitus. J. Pharm Pharmacol 62:1-23.
16. Leonard, B. μL., Watson, R. N., Loomes, K. M., Phillips, A. R., Cooper, G. J. (2005). Insulin resistance in the Zucker diabetic fatty rat: a metabolic characterisation of obese and lean phenotypes. Acta Diabetol. 42: 162-170.
17. Lobmann, R., Ambrosch, A., Schultz, G., Waldmann, K., Schiwecks, S., Lehnert, H. (2002). Expression of matrix-metalloproteinases and their inhibitors in the wounds of diabetic and non-diabetic patients. Diabetologia 45(7):1011-1016.
18. Lobmann, R., Zemlin, C., Motzkau, M., Reschke, K., Lehnert, H. (2006). Expression of matrix metalloproteinases and growth factors in diabetic foot wounds treated with a protease absorbent dressing. J. Diabetes Complicat. 20:329-335.
19. Lowry, O. H., Rosenborough, N. J., Farr, A. μL., Randall, B. J. (1951. Protein measurement. J. Bio. Chem. 193: 265-275.
20. McCarty, S. M., Percival, S. μL. (2013). Proteases and delayed wound healing. Advances in Wound Care, 2(8):438-447.
21. Mast, B. E., Schultz, G. S (1996). Interactions of cytokines, growth factors, and proteases in acute and chronic wound. Wound Repair Regen. 4(4): 411-420.
22. Mignatti, P., Rifkin, D. B., Welgus, H. G., Parks, W. C. (1996). Proteinases and tissue remodeling. In: Clark, R. A. F., ed. The molecular and cellular biology of wound repair. 2^{nd} ed. New York: Plenum Press. 427-474.
23. Milano, S., Arcoleo, F., D'Agostino, P., Cilarri, E. (1997). Intraperitoneal injection of tetracyclines protects mice from lethal endotoxemia down regulating inducible nitric oxide synthase in various organs and cytokine and nitrate secretion in blood. Antimicrob Agents Chemother 41(1): 117-121.
24. Nath, C., Gulati, S. C. (1998). Role of cytokines in healing chronic skin wounds. Acta Haematol 99:175-179.
25. Neely, A. N., Clendening, C. E., Gardner, J. Greenhalgh, D. G. (2000). Gelatinase activities in wounds of healing-impaired mice versus wounds of non-healing-impaired mice. J. Burn. Care Rehabil. 21: 395-402.
26. Neuman, R. E., Logan, M. A. (1950). The determination of hydroxyproline. J. Bio. Chem. 184: 299-306.
27. Papich, M. G., Davidson, G. S., Fortier, μL. A. (2013). Doxycycline concentration over time after storage in a compounded veterinary preparation. J. Am. Vet. Med. Assoc. 242 (12): 1674-1678.
28. Peterson, R. G. (2007). The Zucker diabetic fatty (ZDF) rat-lessons from a leptin receptor defect diabetic model. In: Shafrir, E. editor. Animal models of diabetes: frontiers in research. Boca Raton, Fla.: CRC Press. 103-118.
29. Poonawala, T., Levay-Young, B. K., Hebbel, R. P. (2005). Opioids heal ischemic wounds in the rat. Wound Repair Regen. 13:165-174.
30. Ramachandran, A., Snehalatha, C., Shetty, A. S., Nanditha, A. (2012). Trends in prevalence of diabetes in Asian countries. World J Diabetes. 3:110-117.
31. Ryan, M. E., Baker, D. M. (2001). Tetracycline treatment of periodontal disease: antimicrobial and non-antimicrobial mechanisms. In: Nelson, M., Hillen, W., Greenwald, R. A., eds. Tetracyclines in Biology, Chemistry and Medicine. Basel, Switzerland: Birkhauser-Verlag; 2001; 237-265.
32. Serra, R., Gallelli, μL., Buffone, G., Molinari, V., Stillitano, D. M., Palmieri, C. and de Franciscis, S (2015). Doxycycline speeds up healing of chronic venous ulcers. International Wound Journal. 12:179-184.
33. Shaw, J. E., Sciree, R. A., Zimmet, P. G. (2010). Global estimates of prevalence of diabetes for 2010 and 2030. Diabetes Res Clin Pract. 87:4-14.
34. Sibbald, R. G., Woo, K. Y. (2008). The biology of chronic foot ulcers in persons with diabetes. Diabetes Metab Res Rev 24 (Suppl 1):25-30.
35. SivaNaga, S. A., Anupa, R. M., Manjeet, D., Donald, G., Stanley, S., Jeffrey, μL., Patrick, J. S (2011). Doxycycline hydrogels with reversible disulfide crosslinks for dermal wound healing of mustard injuries. Biomaterials, 32(4), 1204-1217.
36. Smith, G. N., Hasty, K. A. (2001). Structure/function studies of doxycycline effects on matrix metalloproteinase activity and cartilage degeneration. In: Nelson, M., Hillen, W., Greenwald, R. A., eds. Tetracyclines in Biology, Chemistry and Medicine. Boston: Birkhauser-Verlag; 2001; 283-293.
37. Stadelmann, W. K., Digenis, A. G., Tobin, G. R. (1998). Physiology and healing dynamics of chronic cutaneous wounds. The American Journal of Surgery. Vol 176. No. 2, Supplement 1, 26-38.
38. Toker, S., Gulcan, E., Cayc, M. K., Olgun, E. G. Erbilen, E., Ozay, Y. (2009). Topical atorvastatin in the treatment of diabetic wounds. Am. J. Med. Sci. 338: 201-204.
39. Trengove, N. J., Stacey, M. C., MacAuley, S (1999). Analysis of the acute and chronic wound environments: the role of proteases and their inhibitors. Wound Repair Regen. 7: 442-452.
40. Weckroth, M., Vaheri, A., Lauharanta, J., Sorsa, T., Konttinen, Y. T. (1996). Matrix metalloproteinases, gelatinenase and collagenase, in chronic leg ulcers. J. Invest. Dermatol. 106: 1119-1124.
41. Whiting, D. R., Guariguata, μL., Weil, C., Shaw, J. (2011). IDF diabetes atlas: global estimates of prevalence of diabetes for 2011 and 2030. Diabetes Res Clin Pract. 94:311-321.
42. Williams, R. μL., Armstrong, D. G. (1998). Wound healing: new modalities for a new millennium. Clin Podiatr Med Surg 15: 117-128.
43. Wong, V. W., Sorkin, M., Glotzbach, J. P., Longaker, M. T., Gurtner, G. C. (2011). Surgical approaches to create murine models of human wound healing. J. Biomed. Biotechnol. 969618.
44. Woo, K., Ayello, E. A., Sibbald, R. G. (2007). The edge effect: current therapeutic options to advance the wound edge. Adv Skin Wound Care 20: 99-117.
45. Yue, D. K., Swanson, B., McLennan, S. V. (1986). Abnormalities of granulation tissue and collagen formation in experimental diabetes, uraemia and malnutrition. Diabet Med. 3:221-225.
46. Yue, D. K., McLennan, S. V., Marsh, M. (1987). Effects of experimental diabetes, uremia and malnutrition on wound healing. Diabetes 36:295-299.
47. Xu, μL., McLennan, S. V., Lo, μL. (2007). Bacterial load predicts healing rate in neuropathic diabetic foot ulcers. Diabetes Care. 30: 378-380.

1. A transdermal formulation comprising,
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid, at least one antioxidant and a source of doxycycline; and optionally
(d) at least one preservative phase.

2. The transdermal formulation of claim 1, wherein the aqueous phase further comprises arginine, ornithine or an analog thereof.

3. The transdermal formulation of claim 2, wherein the at least one antioxidant is an ascorbic acid ester.

4. The transdermal formulation of claim 1, wherein the source of doxycycline is doxycycline hyclate.

5. The transdermal formulation of claim 1, wherein doxycycline is present in the formulation in an amount of about 0.5 wt % to about 5 wt %, or about 1 wt % to about 3 wt %, of the total formulation.

6. The transdermal formulation of claim 1 in the form of a cream, gel, liquid suspension, ointment, solution or patch.

7. The transdermal formulation of claim 1, in the form of a cream.

8. The transdermal formulation of claim 7, wherein the cream has a viscosity of about 2000 cps to about 500000 cps, or about 5000 cps to about 200000 cps as measured using a Brookfield RVT T4-2, T4-10 or T4-100 RPM instrument at room temperature.

9. The transdermal formulation of claim 1, wherein the formulation maintains its initial color for at least one month.

10. The transdermal formulation of claim 1, wherein the formulation is stable for at least about 20 days at a temperature between about 0° C. to about 25° C., wherein at least about 80% of the doxycycline remains in the formulation after the at least about 20 days.

11. The transdermal formulation of claim 1, wherein the external phase further comprises an essential mineral which is magnesium citrate, magnesium taurate, magnesium glycinate, magnesium glutamate, magnesium aspartate, magnesium chloride, magnesium carbonate or magnesium sulfate.

12. The transdermal formulation of claim 1, wherein the external phase further comprises α-cyclodextrin, β-cyclodextrin or γ-cyclodextrin.

13. A method for the transdermal administration of doxycycline comprising administering an effective amount of one or more of the formulations of claim 1 to a subject in need thereof.

14. A method for treating a doxycycline-responsive disease or condition comprising administering an effective amount of one or more of the transdermal formulations of claim 1 to a subject in need thereof.

15. The method of claim 14, wherein the doxycycline-responsive disease or condition is selected from one or more of diseases and conditions are selected from one or more of Gram-negative and Gram-positive bacterial infections, skin diseases such as acne and rosacea, wrinkles, scleroderma, hyperpigmentation, Clostridium difficile colitis, early Lyme disease, malaria, chronic prostatitis, sinusitis and dermal wound healing.

16. The method of claim 15, wherein the dermal wounds are selected from chronic venous leg ulcers or diabetic foot ulcers.
